(19) United States
(12) Patent Application Publication
(10) Pub. No.: US 2008/0262555 A1
(43) Pub. Date: Oct. 23, 2008

(54) Percutaneous delivery of facet screws using depth control indicator

(75) Inventors: Robert L. Assell, Wilmington, NC (US); Thomas M. Womble, Leland, NC (US)

Correspondence Address: THE ECLIPSE GROUP LLP, 10605 BALBOA BLVD., SUITE 300, GRANADA HILLS, CA, 91344, US

(73) Assignee: TranS1 Inc., Wilmington, NC (US)

(21) Appl. No.: 12/008,111

(22) Filed: Jan. 7, 2008

Related U.S. Application Data
(60) Provisional application No. 60/878,955, filed on Jan. 5, 2007.

(51) Int. Cl. A61B 17/68; A61B 17/58; A61B 17/32

(52) U.S. Cl. 606/301; 606/83; 606/96

(57) ABSTRACT

Instrumentation systems that facilitate the reproducible deployment and placement of fixation device such as a screw, via an aligned, percutaneous access and approach, designed to relieve lower back pain and possibly improve disc health and prevent progression or transition of disease. Information about methods and equipment for accessing and preparing bone for subsequent delivery of a bone screw such as a facet screw across a facet joint. Some of the many teachings contained herein address a system of visual indicators may be used in connection with a bore cutter that operates around an anchored guide wire to avoid dislodging the anchored guide wire.

This application claims priority and incorporates by reference U.S. Provisional Application No. 60/878,955 filed Jan. 5, 2007 for Method and Apparatus for Spine Stabilization Systems.

This application may be used in combination with co-pending and commonly assigned U.S. patent application Ser. No. 11/593,445 filed Nov. 6, 2006 for Application of Therapy Aligned to an Internal Target Path and published as Application No. 2007/0112351. The '445 application as published is incorporated by reference herein.

While one or more applications have been incorporated by reference to provide additional detail it should be noted that these other applications (including any that have subsequently issued as patents) were written at an earlier time and had a different focus from the present application. Thus, to the extent that the teachings or use of terminology differ in any of these incorporated applications from the present application, the present application controls.

### BACKGROUND

### Field of the Disclosure

This disclosure relates generally to medical procedures and in particular, minimally invasive medical procedures. One application of the present disclosure is in providing therapy to adjacent spinal vertebrae. More specifically, one application uses instrumentation systems that facilitate the reproducible deployment and placement of fixation device such as a screw, via an aligned, percutaneous access and approach, designed to relieve lower back pain and possibly improve disc health and prevent progression or transition of disease. This present disclosure provides information about methods and equipment for accessing and preparing bone for subsequent delivery of a bone screw such as a facet screw across a facet joint, or a pedicle screw (including but not limited to trans facet; translaminar, or transpedicular screws). One use of the instrumentation system of the present disclosure is to allow surgeons to accurately and reproducibly deploy, fixation devices such as translaminar facet screws across the facet joints to affix adjacent vertebrae in via a minimally invasive, percutaneous approach, that is, the placement of screws either directly across the facet joints of adjacent vertebrae across the facet joints through the lamina (translaminar) as both a primary mechanism for spinal fixation and as a secondary mechanism for fixation to augment anterior fusion or pedicle screw fixation instrumentation. In addition to the effective and safe placement of devices such as the delivery of facet screws for fusing adjacent vertebrae in a minimally invasive procedure that saves time during surgery and is less traumatic to the patient, as will be understood by one of skill in the art, these instrumentation systems are also applicable to and may facilitate deployment of a variety of other orthopedic devices or screws in motion segments other than or in addition to L5-S1 (for example as used in multi-level therapies), and in the different parts of the spine (such as, cervical; thoracic, with concomitant adjustments as needed and appropriate to system instrumentation dimensions, such as, lengths; diameters) as well as other bones, such as, appendicular surgery or surgery to the foot or the wrist.

Terminology

In the context of the present disclosure anterior refers to in front of the spinal column (ventral); posterior refers to behind the column (dorsal); cephalad means towards the patient's head (also sometimes “superior,”); caudal refers to the direction or location that is closer to the feet (also sometimes “inferior,”). The terms proximal and distal are defined with respect to the surgeon performing the operation. Thus, with respect to components used by the surgeon, the end of a component that is normally held by or at least closer to the surgeon is proximal and the end of a component that is placed into a patient or is most distant to the surgeon during use is distal.

Guide Wire and Guide Pin

While it is common in the medical arts to call items with a diameter of about 1.5 millimeters or less guide wires and items with diameters of about 1.5 millimeters or more guide pins, that distinction is not useful with respect to the present disclosure as the range of diameters that may be used for the guide wires described below may be below or above the approximate 1.5 millimeter cusp. Thus the term guide wire as used herein is not limited to items with a diameter of 1.5 millimeters or less. Sometimes a guide pin is cannulated and sometimes it is not depending on the intended use of the guide pin. As used in herein, the terms guide wire and guide pin may be used interchangeably.

In the context herein, “biocompatible” refers to an absence of chronic inflammation response when or if physiological tissues are in contact with, or exposed to (e.g., wear debris) the materials and devices of the present disclosure.

Items made in accordance with the present disclosure will be manufactured. Any manufacturing process is going to have some range for actual values for any given target value. Many processes have looser tolerances for parameters that are not critical and thus the range of values that may occur in the normal course of manufacturing is broader. Unless otherwise explicitly indicated any dimension provided in this disclosure is to be taken in that context and not as an absolute value. To help illustrate this concept, some of the figures provided with this disclosure include examples of dimensions and manufacturing tolerances for various components (dimensions not marked with units are generally in inches). Further, it is frequently true that sizes are for components are actually nominal sizes rather than precise sizes.

Introduction to Relevant Anatomy and Terms

The spinal column is a complex system of bone segments (vertebral bodies and other bone segments) which are in most cases separated from one another by discs in the intervertebral spaces (sacral vertebrae are an exception). In the context of the present disclosure, a “motion segment” includes adjacent vertebrae, (an inferior and a superior vertebral body), and the intervertebral disc space separating said two vertebral bodies (discussed below). Unless previously fused, each motion segment contributes to the overall flexibility of the spine. In other words, unless previously fused, each motion segment contributes to the overall ability of the spine to flex to provide support for the movement of the trunk and head.

The individual motion segments within the spinal columns allow movement within constrained limits and provide protection for the spinal cord. The discs are important to cushion and distribute the large forces that pass through the spinal column as a person walks, bends, lifts, or otherwise moves. Unfortunately, for a number of reasons, for some people, one or more discs in the spinal column will not operate as intended. The reasons for disc problems range from a congenital defect, disease, injury, or degeneration attributable to aging. Often when the discs are not operating properly, the gap between adjacent vertebral bodies is reduced and this causes additional problems including pain.

Anatomy of a Motion Segment

Turning to FIGS. 1 and 2, the components of the vertebrae are introduced. FIG. 1 is a top perspective view of a single vertebra 704. The vertebra has a hard outer shell of cortical bone 784 and an interior of cancellous bone 788.

The spinal cord (not shown) is protected in the spinal foramen 792 formed by the two pedicles 712, 716 and the two laminae 720, 724. Extending from the pedicles are two transverse processes 728, 732. Extending from the midline of the vertebra where the two laminae meet is the spinous process 736. These three processes serve as connection points for ligaments and muscle.

Vertebrae move relative to one other in order to allow the spine to bend forward (flexion), bend backward (extension), bend to the right or left (lateral bending), twist (rotate in the z-axis) and other forms of movement. While the disc 780 plays an important part in this movement in absorbing shocks and distributing loads, there are also joints on the posterior side of the spinal column that allow for movement of a vertebra relative to an adjacent vertebra.

These joints are called facet joints. Most vertebrae have four facet joints. Two facet joints between a particular vertebra and the adjacent cephalad vertebra and two facet joints between the particular vertebra and the adjacent caudal vertebra.

The components of the facet joints are the superior articular process 740 and 744 and the inferior articular process 748 and 752.

FIG. 2 is a rear perspective view of a motion segment 700 with a lower (more caudal) vertebra 704 and a higher (more cephalad) vertebra 804. The anterior portion of the vertebra is the vertebral body 708, 808. Between the two vertebral bodies 708 and 808 is a disc 780. The spinous processes 736 and 836 and the transverse processes 728, 732, 828, 832 are visible in this view.

The facet joint portion of the superior articular processes 740 and 744 for vertebra 704 are engaged by the inferior articular processes 848 and 852 of vertebra 804 to as part of facet joints 742 and 746. The superior articular processes 840, 844 for the vertebra 808 are visible as they would engage with the inferior articular processes from the next more cephalad vertebra. Likewise the inferior articular processes 748, 752 of vertebra 704 would engage with the superior articular processes of the next more caudal vertebra. A neuralforamen 856 (sometimes neural foramen) is partially visible in FIG. 2. There is another neuralforamen on the opposite side. The neuralforamina provide a passage for the nerves connecting to the spinal cord. If this passage way is constricted, the constriction called stenosis of the neuralforamina can cause pain or other neural symptoms.

With respect to motion, vertebrae move relative to one other in order to allow the spine to bend forward (flexion), bend backward (extension), bend to the right or left (lateral bending), twist (rotate in the z-axis) and other forms of movement. While the disc plays an important part in this movement in absorbing shocks and distributing loads, there are also joints on the posterior side of the spinal column that allow for movement of a vertebra relative to an adjacent vertebra.

These joints are called facet joints. Most vertebrae have four facet joints. Two facet joints between a particular vertebra and the adjacent cephalad vertebra and two facet joints between the particular vertebra and the adjacent caudal vertebra. The components of the facet joints are the superior articular process the inferior articular process. The facet joints are positioned between each pair of adjacent vertebrae, share and support with the respective intervertebral disc included in that motion segment, compressive axial loads on the spine. Spinal mobility intricately involves movement of the motion segment and the loss of normal movement of the motion segment resulting from the loss of disc hydration. Specifically, fluidity, elasticity and thickness of the disc are important for mobility. With the loss of disc hydration, the discs lose some of their ability to act as a cushion. The structures comprising the motion segments of the spine undergo a deformative process, since with the loss of hydrostatic pressure within the disc; the disc loses its viscoelastic capacity to attenuate shock and can no longer uniformly distribute loads. As the nucleus pulposus loses its water content, the disc collapses, resulting in a narrowing of the intervertebral disc space and causing the two vertebrae above and below to move closer to one another. As this shift occurs, the facet joints located on the posterior column of the spine are forced to shift. This loss of disc height often also alters the facet joint's mechanical ability. (Those with an interest in additional drawings of the anatomy of facet joints are directed to FIGS. 1a-1d of the provisional 60/878,955 referenced above and available through Public PAIR at www.uspto.gov after this application is published).

Thus, it is also known to place fixation devices such as screws, either directly across the facet joints of adjacent vertebrae or indirectly across the facet joints through the lamina (i.e., translaminar) as a primary mechanism for spinal fixation and also as an ancillary mechanism for fixation to augment anterior fusion or pedicle screw fixation instrumentation, and as such, both direct and translaminar facet screws are often being implanted. More specifically, translaminar, transarticular, transpedicular, and/or transfacetpedicular bone fasteners (e.g., screws, pins, wires) inserted contralaterally or ipsilaterally are sometimes used alone or in combination/adjunct with other devices in order to create an anterior/posterior fixation construct as a mechanical, load-sharing aid to fracture healing or interbody (e.g., L5-S1) stabilization and fusion of the lumbar spine.

The fixation devices and their deployment by means of the instrumentation system of the present disclosure enable an effective adjunct therapy to procedures such as anterior lumbar interbody fusion (ALIF); transforaminal lumbar interbody fusion (TLIF); laminectomy; laminotomy; foraminotomy; discectomy, including anterior cervical discectomy and fusion (ACD; ACDF). More specifically, yet another advantage of the system of the present disclosure is the biomechanical “balance” derived from an ability to deploy the posterior stabilizing devices in conjunction with an anterior supporting structures, e.g., axially deployed implants such as those described in commonly assigned U.S. Pat. No. 6,921,403 issued Jul. 26, 2005 and U.S. application Ser. No. 11/202,655 filed Aug. 13, 2005, deployed by means of instrumentation such as described in co-pending and commonly assigned U.S. patent application Ser. Nos. 10/971,779; 10/971,781; 10/971,731; 10/972,077; 10/971,765; 10/972,065; 10/971,775; 10/972,299; 10/971,780 each filed on Oct. 22, 2004 and 11/501,351 filed Aug. 9, 2006, the contents of each of the aforementioned U.S. patent applications are hereby incorporated into this disclosure by reference. (These earlier applications are available through the USPTO and may be accessed through Public PAIR at www.uspto.gov).

Thus, the teachings of the present disclosure may be used as an adjunct to, for example, anterior column disc or nucleus pulposus replacement (procedures which involve the removal of tissue and which destabilize the spine) as part of a fusion procedure, in addition to primary stand-alone treatment for patients with isolated posterior column disease as noted above to maintain the mechanical relationship between the anterior and posterior column structures.

The facets are true articulating joints in the lumbosacral spine, and play an important role in guiding segmental motion and in limiting their maximal range, so it is logical to fix these directly (i.e., one therapeutic treatment of the facet joint is to affix the superior articular process to the inferior articular process using a facet screw) to achieve spine fixation. Facet screws may be inserted bilaterally through the superior side of the facet, across the facet joint, and into the pedicle. Alternatively, the facet screws may be inserted from the base of the spinous process into the opposite lamina and across the facet joint and into the base of the lower vertebral transverse process. Lower lumbar spinal fusion in patients by techniques involving screw fixation of the facet joints often achieves stability comparable to that of pedicle screw fixation even after long-term repetitive cycling, although pedicle screw systems are generally used in lieu of facet screws when there is degenerative disease of the facets with instability. Bone quality may dictate the fixation method.

Such therapies may be indicated to treat pseudoarthrosis, spinal stenosis, spondylolisthesis, segmental degenerative disease, or degenerative disc disease, and supplementation of posteriolateral fusion by means of such screws may significantly improve time to fusion, fusion rate, and clinical outcomes.

Orthopaedic Drill Bits

Of relevance to evaluation of the novel cutting tool disclosed below is the extent to which the prior art teaches away from the teachings of the present disclosure. Thus, it is useful to review the features of standard orthopedic devices in the prior art. Such devices, or drill bits, are typically constructed with a cutting end and a shaft characterized with a spiral, or helix. As depicted in FIG. 3, the cutting end (distal end) of a generic cutting tool 104 has a cutting edge 108 moving in a direction of cutting 112. The cutting edge 108 may be characterized by a rake angle 116 and the clearance angle 124. The wedge angle 120 is that between the cutting face 128 and the flank 132, while the rake angle 116 is the angle at which the cutting face 128 is presented to the material. The clearance angle 124 is the angle by which the flank 132 of the drill clears the material that has been cut. These terms for all cutting tools apply generally to drill bits.

Moving to FIG. 4a-4c, the tip of a drill bit 152 is shown in FIG. 4a in profile, from a front view in FIG. 4b, and a portion of a drill bit is shown to illustrate the helix, wedge, and clearance angels. The drill bit 152 can be further described in terms of a point angle 136, the flank 132 and the chisel edge 144. The point angle 136 is that angle between the two cutting edges measured in a sagittal plane and in standard orthopedic drill bits is typically about 90 degrees.

With reference to prior art drills, the flank 132 is the flat part of the drill bit when viewed end-on (FIG. 4b); this flank surface is not totally in contact with the bone surface, but separated from it by the clearance angle 124 (FIG. 4c). The flank 132 of the drill bit 152 represents a large surface area for friction and the generation of heat. The term chisel edge 144 refers to the tip of the drill bit 152 at the apex of the point angle 136. The helix of a drill bit 152 is measured as the helix angle 148 (FIG. 4c) and is often defined as slow, standard or quick, representing small, standard and large helix angles, respectively. A quick helix has more turns per unit length than a slow helix. Traditionally, a slow helix is used to drill material from which debris clears easily, such as brass or cast iron, in conjunction with a large point angle, while fast helix would be preferable for materials which must be cleared quickly to avoid blocking the flutes 140, and attempts have been made to pattern flutes (e.g., parabolic) or alter helix lengths to improve clearance of debris from deep holes, such as cortices of a femur, and allow higher feed rates of drill bit advancement.

Moreover, there are studies in the literature which appear to suggest that for orthopedic drill bits, substantially large point angles are both needed and preferred for optimum performance (e.g., Saha et al., [1982] and Natali, et al., [1996] recommend 118° while Fuchsberger [1987] recommends 70° to 75°.

References may be found at: 1) Bonfield W, and C H Li. The temperature dependence of the deformation of bone. J Biomechanics 1968; 1:323-9. 2) Fuchsberger A. Optimization of the spiral drill for use in medicine. Z Orthop 1987; 125:290-7. 3) Natali C., Ingle, P., and J. Dowell. The Journal of Bone and Joint Surgery VOL. 78-B, No. 3, MAY 1996 p. 357-362. 4) Saha S., Pal S., and J A Albright. Surgical drilling: design and performance of an improved drill. J Biomech Eng 1982; 104:245-52.

### SUMMARY

Aspects of the teachings contained within this disclosure are addressed in the claims submitted with this application upon filing. Rather than adding redundant restatements of the contents of the claims, these claims should be considered incorporated by reference into this summary.

This summary is meant to provide an introduction to the concepts that are disclosed within the specification without being an exhaustive list of the many teachings and variations upon those teachings that are provided in the extended discussion within this disclosure. Thus, the contents of this summary should not be used to limit the scope of the claims that follow. Other systems, methods, features and advantages of the disclosed teachings will be or will become apparent to one with skill in the art upon examination of the following figures and detailed description. It is intended that all such additional systems, methods, features and advantages be included within the scope of and be protected by the accompanying claims.

### BRIEF DESCRIPTION OF THE FIGURES

The disclosure can be better understood with reference to the following figures. The components in the figures are not necessarily to scale, emphasis instead being placed upon illustrating the principles of the disclosure. Moreover, in the figures, like reference numerals designate corresponding parts throughout the different views.

FIG. 1 is a top perspective view of a single vertebra 704.

FIG. 2 shows a pair of vertebrae from a rear perspective view.

FIG. 3 shows concepts for generic cutting tools.

FIG. 4a-4c shows concepts of prior art drill bits.

FIG. 5 is a flow chart showing one method of deploying facet screws.

FIG. 6 introduces a set of instruments for use in the method described in FIG. 5.

FIG. 7 illustrates a rear view of the lower spine with a trajectory for placement of a facet screw across a L5/S1 facet joint.

FIG. 8 provides an enlarged view of the distal end of the guide pin assembly at the facet joint.

FIG. 9 shows an enlarged view of the visual indicia section 2052 of the guide wire 2040.

FIG. 10 is a side view of a fully threaded facet screw.

FIG. 11 is a side view of a lag facet screw.

FIG. 12 shows a longitudinal cross section of a fully threaded facet screw 2400.

FIG. 13 provides an enlarged view of detail C from FIG. 12.

FIG. 14 shows a front view of the distal end 2404 of the fully threaded facet screw.

FIG. 15 shows a side view of the cannulated guide pin 2008.

FIG. 16 shows the details of the distal end 2024 of the cannulated guide pin 2008.

FIG. 17 shows details of the proximal end 2020 of the cannulated guide pin 2008.

FIG. 18 shows a side view of the guide pin with grip 2016.

FIG. 19 provides detail on the grip 2150.

FIG. 20 shows a side view of the subassembly of the cannulated guide pin 2008 with inserted guide pin with grip 2016.

FIG. 21 is a cross section of the complete guide pin assembly.

FIG. 22 is an enlarged detail of FIG. 21.

FIG. 23 shows the first grind which puts a point angle of about 30 degrees on the guide wire

FIG. 24 shows the second grind at about 15 degrees to form a cutting point on the guide wire.

FIG. 25 is a cross section of the dilator sheath 2084.

FIG. 26 provides details of the proximal end 2096 of the dilator sheath 2084.

FIG. 27 provides details of the distal end 2092 of the dilator sheath 2084.

FIG. 28 provides a perspective view of the distal end 2204 of the bore cutter 2200.

FIG. 29 provides a side view of details of the bore cutter.

FIG. 30 shows a front view of the bore cutter 2200 from FIG. 28.

FIG. 31 is a cross section of the bore cutter taken to show the clearance angle 2240.

FIG. 32 provides a top view of a bore cutter 2200.

FIG. 33 is a perspective view of the distal end of bore cutter 2200 with the distal end 2044 of the guide wire 2040.

FIG. 34 shows a top view of a facet screw tap 2110.

FIG. 35 is an enlarged perspective view of the distal end of the facet screw tap.

FIG. 36 shows a L5/S1 motion segment with an implanted fusion rod and one of the two facet screws across one of the two facet joints.

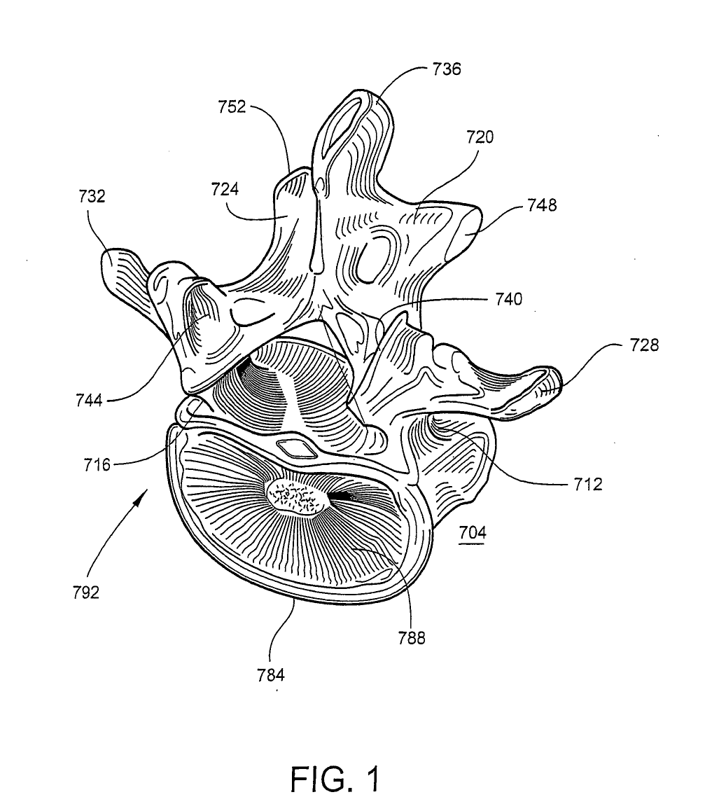

### DETAILED DESCRIPTION

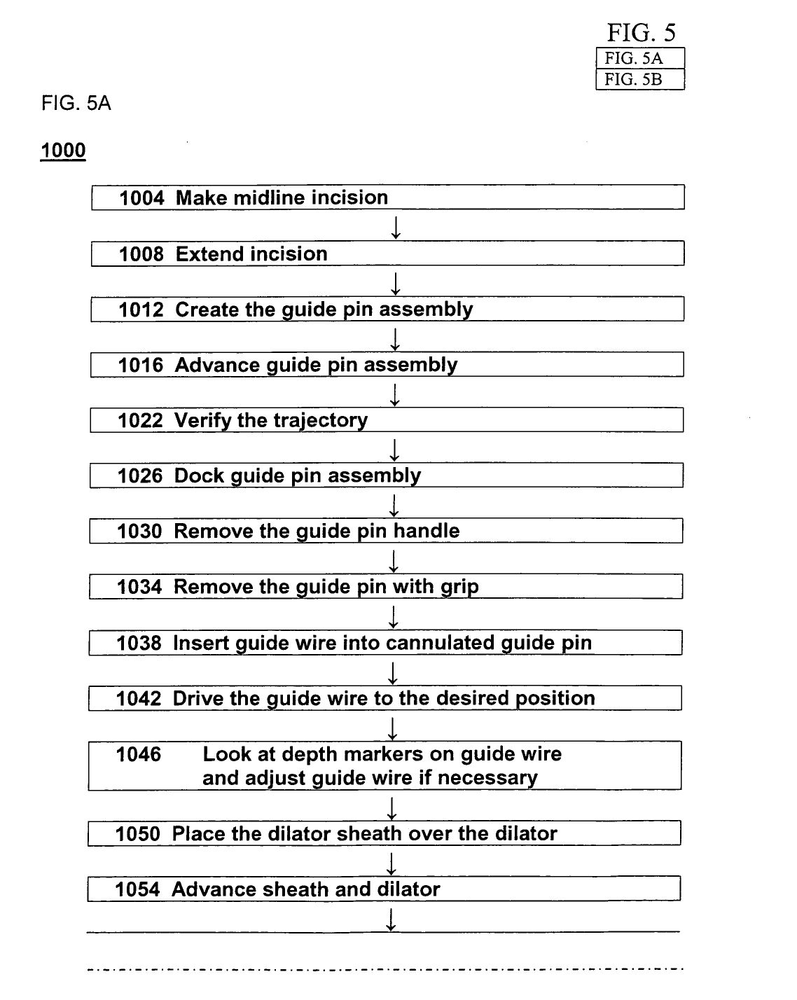

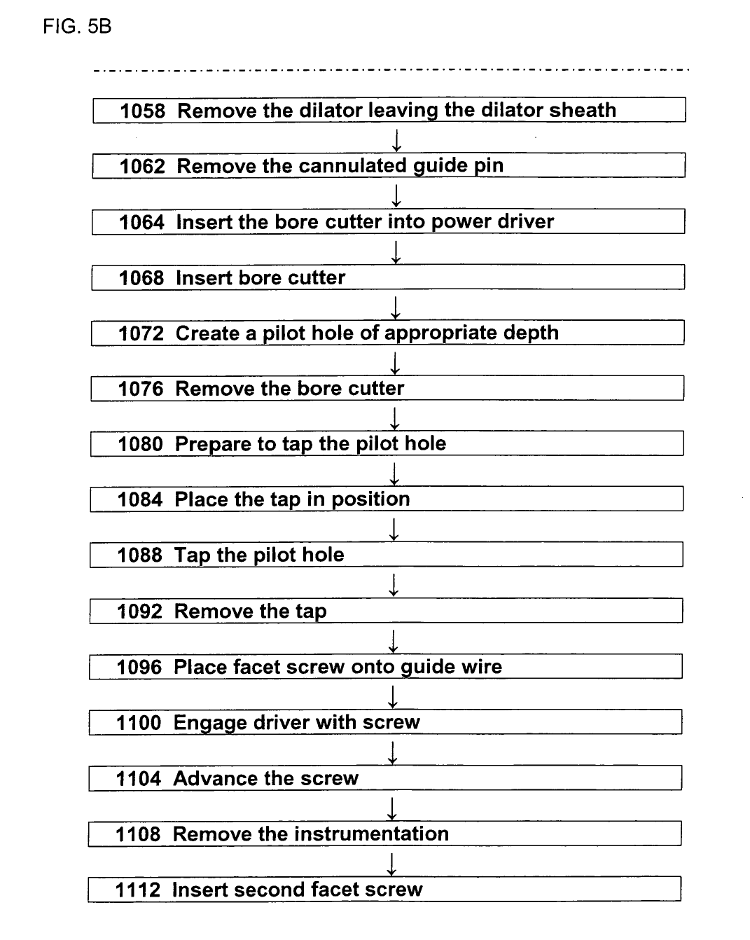

In order to provide context for the various pieces of equipment to be described in detail below, it is useful to start with a description of one exemplary method for delivering a facet screw across a facet joint as set forth in the flow chart in FIG. 5. This particular sequence is adapted for L5/S1 facet joint. This sequence of steps references a set of instruments introduced in FIG. 6. To avoid interruptions in the description of the process, the instruments are described in greater detail in subsequent sections.

To provide context for the process, it is useful to note that the operating room may be set up as follows.

The patient may have received preparation for spine surgery as known in the art. Biplanar fluoroscopy, known in the art, may be used to allow for visualization of instruments that are inserted into the patient. (Biplanar fluoroscopy includes AP (anterior/posterior) and lateral fluoroscopy.) Note that a surgeon may opt to use one fluoroscope and have it moved from the one plane to another or alternatively the surgeon may have two fluoroscopes set up, one in each plane. Having two scopes set up saves the time involved in moving the scopes back and forth but some surgeons prefer to have less equipment as the equipment takes up space.

The patient may be placed in a prone position. The patient's lumbar spine will be in flexion and hips will be in flexion. Often, the placement of facet screws will follow the insertion of a fusion rod in accordance with teachings in the various applications cited and incorporated by reference into this application.

The AP fluoroscopy may be oriented so that the vertebral endplate of L5 may be viewed from the Ferguson angle. Before proceeding, the surgeon may wish to note the landmarks of the L5/S1 facets and the L3 spinous process.

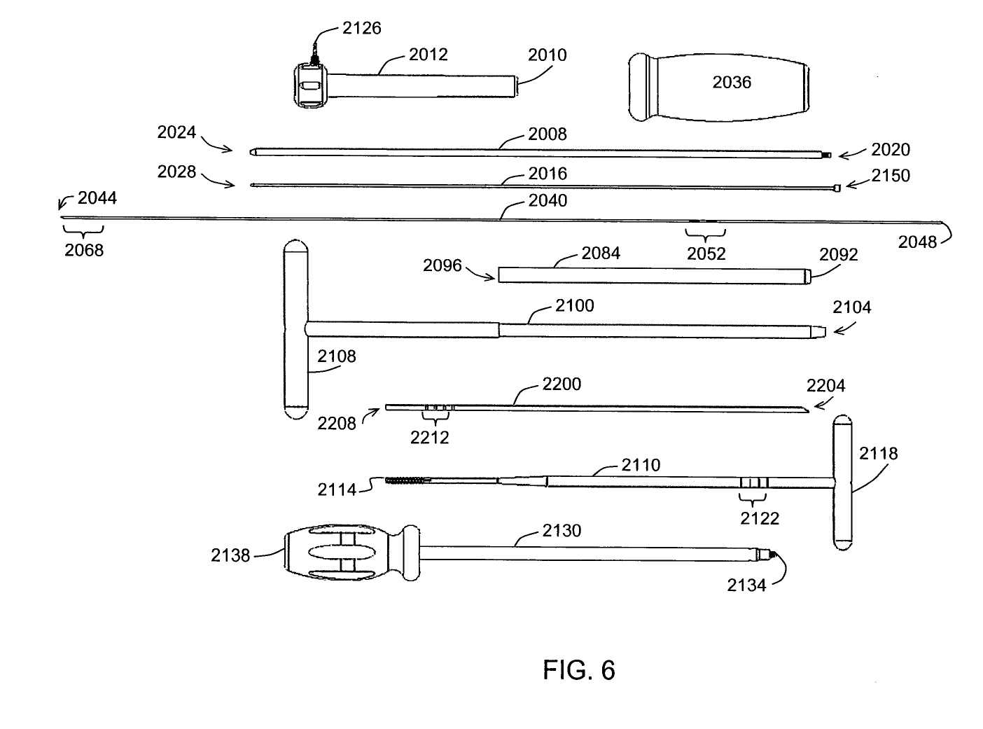

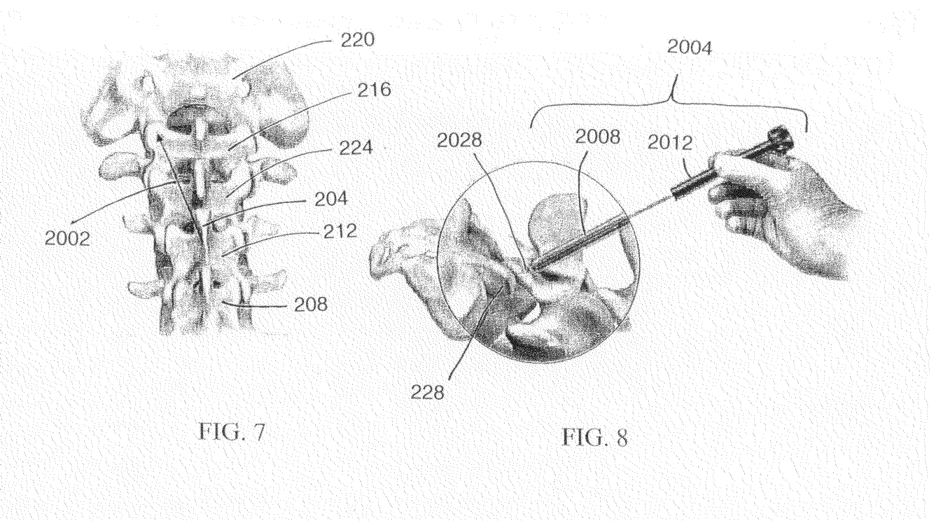

Step 1004 Create incision. As shown in FIG. 7, make a midline incision at the L3 spinous process 204. The midline incision is first made in the skin between the L2 208 and L3 212, or at the L3 spinous process 204. The location of the incision may vary, dependent on individual patient anatomy. FIG. 7 shows the general trajectory 2002 desired for placing a facet screw into one of the facet joints 228 between the L5 216 and S1 220 vertebrae. As it is relevant to the trajectory, the L4 224 vertebrae are also identified in FIG. 7.

Step 1008 Extend incision. Continue the incision through the dorsal facia on either side of the L3 spinous process (the exact position of the incision may vary due to patient anatomy).

Step 1012 Create the guide pin assembly. Turning to FIG. 6 and FIG. 8, create the guide pin assembly 2004 by inserting the distal end 2028 of the guide pin with grip 2016 into the proximal end 2020 of the cannulated guide pin 2008. Place the distal end 2010 of the guide pin handle 2012 over the grip 2150 of the guide pin with grip 2016 and the proximal end 2020 of the cannulated guide pin 2008. Tighten the enlarged set screw 2126 to capture both the proximal end 2020 of the cannulated guide pin 2008 and the grip 2150 of the guide pin with grip 2016.

Step 1016 Advance guide pin assembly. The guide pin assembly 2004 is inserted and advanced until the distal end 2032 contacts the inferior articular process of L5 (superior portion of the L5/S1 facet on this side), as shown in FIGS. 7 and 8. One possible technique is to advance the guide pin assembly 2004 down the lamina of L3 212, L4 224, and L5 216 (See FIG. 7). Another alternative is to advance the guide pin assembly 2004 directly to the facet joint. Percutaneous insertion of the guide pin assembly 2004 may be performed by the surgeon while using radiographic visualization, such as fluoroscopic guidance by means of both anteroposterior (AP) and lateral fluoroscopy to safely enable advancement of the guide pin assembly 2004 through the soft tissue of the posterior access track up to the target site on a spinal vertebra and to locate the facet joint. On the lateral fluoroscope view, a good initial bony landmark to aim at is the midpoint of the inferior process of L5 angling the distal end 2032 of the guide pin assembly 2004 towards the neck of the S1 pedicle. The surgeon moves the guide pin assembly 2004 to laterally angle and advance the distal end 2032 of the guide pin assembly 2004 towards the facet joint 228, so that its distal end 2032 is directly above the center of the facet joint 228 and not into the spinal canal (as best seen in the fluoroscopic AP view). Once the facet joint 228 is located, adjustments are made to the guide pin assembly 2004 via the guide pin handle 2012 to achieve the desired trajectory 2002 towards the pedicle, which is again verified in both AP and Lateral planes using the fluoroscope.

Step 1022 Verify the trajectory in both AP and Lateral Planes using the fluoroscope.

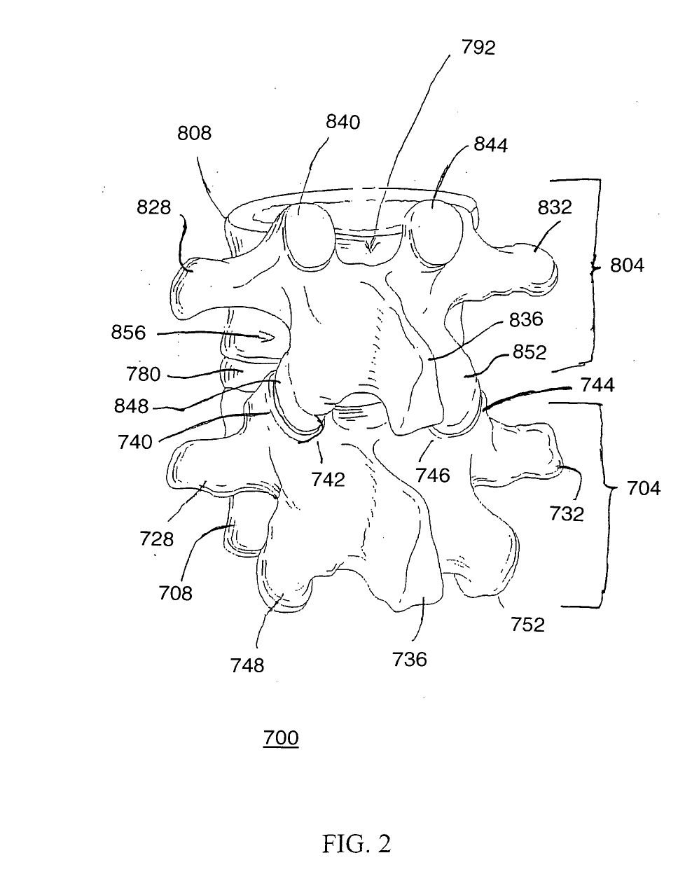

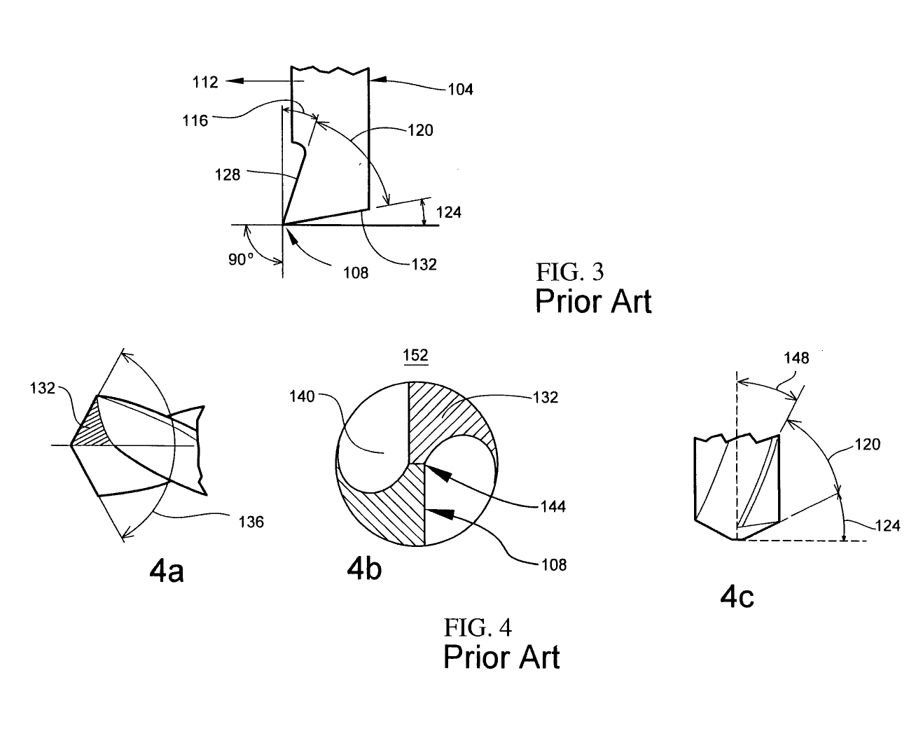

Step 1026 Dock guide pin assembly 2004 into inferior articular process of L5 at facet joint 228 (See FIG. 2 elements 848 and 852 for examples of inferior articular processes). A mallet or other impact delivery tool such as a slap hammer (FIG. 6 element 2036) is then used to dock the cannulated guide pin 2008 into the inferior articular process of L5. The process of lightly docking may move the distal end 2024 of the cannulated guide pin 2008 a few millimeters into the facet joint.

Step 1030 Remove the guide pin handle 2012 from the Guide Pin Assembly removed while leaving in place the cannulated guide pin 2008 and the guide pin with grip 2016 Step 1034 Remove the guide pin with grip 2016 from the cannulated guide pin 2008. This may be done by using a Kelly clamp to twist and remove the guide pin with grip 2016.

Step 1038 Insert the distal end 2044 of the guide wire 2040 into the proximal end 2020 of the cannulated guide pin 2008.

Step 1042 Drive the distal end 2044 of the guide wire 2040 to the desired position. Using a wire driver, the distal end 2044 of the guide wire 2040 is advanced across the facet joint so that it is securely engaged in the facet, while fluoroscopically verifying proper placement and trajectory, and repositioning as needed. The wire driver may actually be the same device that is used to drive the boring cutter. The distal end 2044 of the guide wire 2040 may be ground to provide a drilling tip to allow the guide wire 2040 to be driven rather than a conventional tip at the axial centerline of the guide wire (not shown).

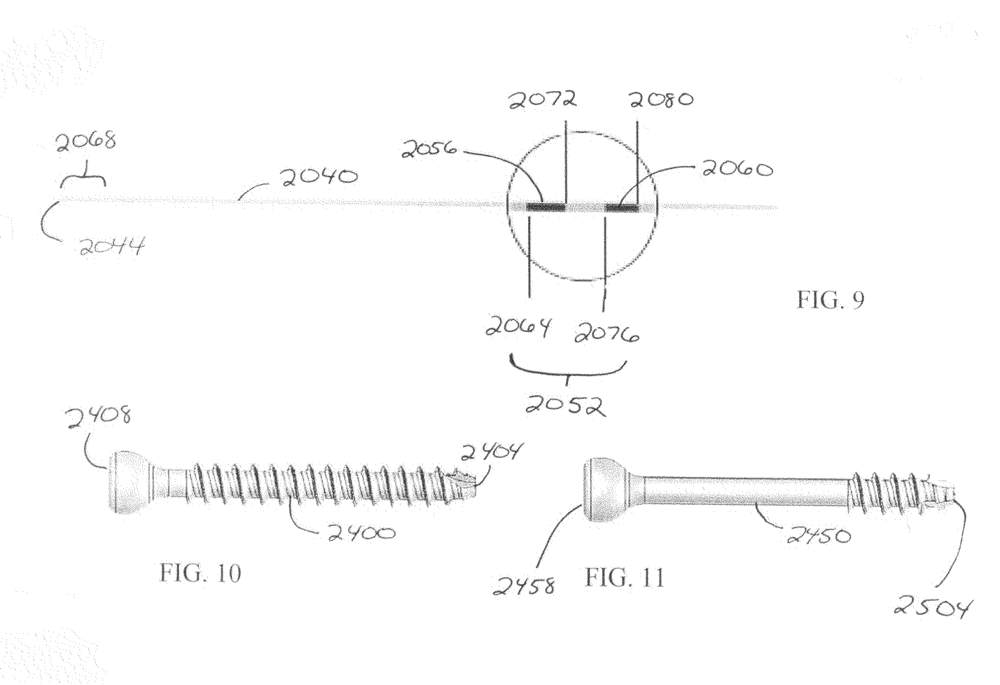

Step 1046 Look at depth markers on guide wire and adjust guide wire if necessary. Once the surgeon has achieved the desired placement, trajectory and depth, the guide wire 2040 is examined to determine the appropriate nominal size of facet screw to use. FIG. 9 show guide wire 2040 with distal end 2044 that is driven across the facet joint and proximal end 2048 that extends beyond the cannulated guide pin 2008. FIG. 9 shows an enlarged view of the visual indicia section 2052 of the guide wire 2040. While the visual indicators could be arranged in any of a number of ways, in this example there are two laser marked bands 2056 and 2060. As the guide wire 2040 is much narrower than the diameter of the bore cutter or tap discussed below, it is thought that using bands rather than individual strips for each landmark makes the visual indicators easier to see.

These bands are placed on the guide wire 2040 so that the surgeon receives an indication of the appropriate nominal screw length to use based on the inserted depth of the distal end 2044 of the guide wire 2040 beyond the distal end 2024 of the cannulated guide pin 2008 (See FIG. 6). For example, when the distal end 2044 of the guide wire 2040 extends a distance of approximately 30 millimeters beyond the distal end 2024 of the cannulated guide pin 2008, the distal edge of the more distal band will be approximately at the proximal end 2020 of the cannulated guide pin 2008. This landmark 2064 will indicate that a nominal length of 25 millimeters will be appropriate as a 25 millimeter bore may be prepared while leaving a suitable length 2068 of the distal end 2044 of the guide wire 2040 anchored so that the process of preparing a bore does not dislodge the guide wire 2040. While other distances may be used, an offset of 5 millimeters between the actual extension of the distal end of the guide wire 2040 and the length of screw suggested by the landmark provides a suitable length 2068 of guide wire 2040 that remains anchored after boring. The width of the bands and the gap between bands provides three other landmarks 2072, 2076, and 2080 to indicate that three other nominal screw lengths are appropriate.

If at the start of step 1046 all of the landmarks for the visual indicia section 2052 are within the cannulated guide pin 2008, then the surgeon may choose the longest nominal screw length (such as 40 millimeters in the present example) and simply have more guide pin anchored after the boring step.

If at the start of step 1046 all of the landmarks for the visual indicia section 2052 are visible and the landmark for the shortest suggested nominal screw length (in this case landmark 2064) are close to the proximal end 2020 of the cannulated guide pin 2008, then the surgeon may drive the guide wire 2040 further into the patient to ensure an adequate insertion depth to allow the guide wire 2040 to remain anchored after preparing a bore for the shortest nominal screw length (25 millimeters in this example).

Step 1050 Place the dilator sheath 2084 over the dilator 2100 if it is not already in place. See FIG. 6 for these components. More specifically, the proximal end 2096 of the dilator sheath 2084 is placed initially over the distal end 2104 of the dilator 2100. This example uses a 6 millimeter dilator.

Step 1054 Advance the sheathed dilator. Advance the distal ends of the Dilator and Dilator Sheath to the facet joint. More specifically, hold the handle 2108 of the dilator 2100 and advance the distal end 2104 of the sheathed dilator 2100 over the proximal end 2048 of the guide wire 2040 and the proximal end 2020 of the cannulated guide pin 2008 and advance the sheathed dilator 2100 to the facet joint.

Step 1058 Remove the dilator 2100 while leaving the inserted dilator sheath 2084 in place. The proximal end 2096 of the dilator sheath 2084 extends out of the patient.

Step 1062 Remove the cannulated guide pin 2008. More specifically, the proximal end 2020 of the cannulated guide pin 2008 extends beyond the proximal end 2096 of the dilator sheath 2084. Thus, the cannulated guide pin 2008 may be grasped and removed from the dilator sheath 2084 while leaving the anchored guide pin 2016. Some may choose to twist the docked cannulated guide pin 2008 while holding the dilator sheath 2084 in place in order to free the docked distal end 2024 of the cannulated guide pin 2008. Alternatively, the guide pin handle 2012 may be re-attached to the cannulated guide pin 2008 so that the guide pin handle 2012 may be used to assist in releasing the docked distal end 2024 of the cannulated guide pin 2008 such as by tapping on the guide pin handle 2012 to move the guide pin handle 2012 away from the facet joint.

Step 1064 Insert the proximal end 2208 of the bore cutter 2200 into a power driver (not shown).

Step 1068 Insert the distal end 2208 of the bore cutter 2200 over the proximal end 2048 of the anchored guide wire 2040 as the guide wire diameter is slightly less than the inner diameter of a cannula that runs through the bore cutter 2200. Advance the distal end 2208 of the bore cutter 2200 into the proximal end 2096 of the dilator sheath 2084 and to the distal end 2092 of the dilator sheath 2084.

Step 1072 Create a pilot hole of appropriate depth. Use the power driver to rotate the bore cutter 2200 to advance the distal end 2204 of the bore cutter 2200 until the appropriate landmark from the set of visual indicators 2212 on the shaft of the bore cutter 2200 approaches the proximal end 2096 of the dilator sheath 2084. The surgeon may wish to view the distal end 2204 of the bore cutter 2200 while it is being advanced using fluoroscopy. The use of fluoroscopy alone may be sufficient to prevent a surgeon from advancing the distal end 2204 of the bore cutter 2200 too deep and thus dislodging the anchored distal tip 2044 of the guide wire 2040. However, the use of the landmarks will allow more precise depth control and facilitate the maintenance of a standardized suitable length 2068 of anchored guide wire 2040.

For example, if at step 1046, the landmark on the guide wire 2040 indicated a 25 millimeter screw was appropriate, advance the bore cutter 2200 to create a bore until the 25 millimeter screw landmark approaches the proximal end 2096 of the dilator sheath 2084. This landmark for a 25 millimeter screw may be established so that the bore cutter 2200 extends approximately 25 millimeters beyond the distal end 2092 of the dilator sheath 2084 to create a pilot bore of approximately 25 millimeters while leaving approximately 5 millimeters of the guide wire 2040 anchored in the pedicle. (5 millimeters being the selected suitable length 2068 of guide wire to be left anchored in this example). Keeping the distal end 2092 of the dilator sheath 2084 abutting the facet joint leads to predictable pilot hole depth. Actually, there is another small amount of bias that helps promote a difference in insertion depth between the distal end of the pilot hole and the distal end of the guide wire 2040 as the guide wire 2040 was inserted a distance beyond the distal end 2024 of the cannulated guide pin 2008 which was itself partially inserted into the facet joint when it was docked. As the distal end 2092 of the dilator sheath 2084 rests against the facet joint and is not docked, the bore cutter 2200 starts its measured extension from the outer surface of the facet joint rather than partially in the facet joint.

The landmarks on the shaft of the bore cutter 2200 may be presented as shown above for the guide wire 2040 (a pair of laser marked bands). Alternatively, there may be a series of landmark rings, one ring for each nominal screw length.

Step 1076 Remove the bore cutter 2200 from the guide wire 2040 while leaving the anchored guide wire 2040 in place and leaving the dilator sheath 2084 in place. Note that it is typical for the pilot hole to have a smaller diameter than the minor diameter of the screw to be used.

Step 1080 Prepare to tap the pilot hole. To tap the pilot hole created by the cutting bore, the facet screw tap 2110 is examined to locate a landmark within a set of visual indicators 2122 near the handle 2118 on the facet screw tap 2110. More specifically, the landmark corresponding to the previously selected nominal screw length is located.

Step 1084 Place the distal end of the tap in position. More specifically, place the distal end 2114 of the facet screw tap 2110 (See FIG. 6) over the proximal end 2048 of the guide wire 2040 and move the distal end 2114 of the facet screw tap 2110 through the dilator sheath 2084 to the start of the pilot hole.

Step 1088 Tap the pilot hole. Using the proximal end 2096 of the dilator sheath 2084 as a guide, advance the tap by rotating the handle 2118 until the appropriate landmark for the selected nominal screw length approaches the dilator sheath 2084. (In this example the tap is operated by rotating the handle 2118 in a clockwise direction to advance it and counterclockwise to remove it but this would be reversed if the handedness of the facet screws was reversed.) The surgeon may occasionally monitor the advancement of the distal end 2114 of the facet screw tap 2110 using fluoroscopy.

If a 25 millimeter nominal screw length was indicated back in step 1046, then the pilot hole would have a depth of approximately 25 millimeters but the distal end 2044 of the anchored guide wire 2040 would extend beyond the distal end 2092 of the dilator sheath 2084 approximately 30 millimeters so that the creation of the pilot hole and the subsequent tapping did not disturb approximately 5 millimeters of guide wire 2040 anchored in the pedicle. The tap may be selected so as to tap threads that correspond to a major diameter that is less than the major diameter to be found on the selected facet screw. This is known as under-tapping and allows the facet screw to cut it own thread path while benefiting from the previously tapped pilot hole.

Step 1092 Remove the tap. Rotate the handle 2118 the opposite direction from that used to advance the facet screw tap 2110 to remove the facet screw tap 2110 and then pull the facet screw tap clear of the dilator sheath 2084 and guide wire 2040.

Step 1096 Place the facet screw onto the guide wire. Surgeons may select a fully threaded facet screw 2400 (FIG. 10) or a lag facet screw 2450 (FIG. 11). These screws will be described in more detail below. The screws are available in a set of nominal lengths corresponding the landmarks on the guide wire 2040, bore cutter 2200, and facet screw tap 2110. With either screw, place the distal end 2404 or 2454 of the cannulated facet screw 2400 or 2450 over the proximal end 2048 of the guide wire 2040 and advance the screw onto the guide wire 2040.

Step 1100 Engage the driver with the screw. Place the cannulated distal end 2134 of the driver 2130 (FIG. 6) over the proximal end 2048 of the guide wire 2040 and engage the distal end 2134 of the driver 2130 with a corresponding section in the proximal end 2408 of fully threaded facet screw 2400 or proximal end 2458 of lag facet screw 2450. Typically, the driver would have a hex tip (for example a 3 millimeter hex tip) to fit into a corresponding hex opening but one of skill in the art could use a square driver, Phillips head driver or some other driver. While a T-handle similar to that used with the facet screw tap 2110 could be used at the proximate end 2138 of the driver 2130, a screw driver type handle as shown in FIG. 6 may be preferred as in some instances there may be insufficient clearance between a T handle and the exterior of the patient.

Step 1104 Advance the screw in the pilot hole. Rotate the driver 2130 the appropriate direction for the thread handedness, to advance the facet screw (2400 or 2450) and engage the threads of the screw with the walls of the pilot hole. After the screw (2400 or 2450) is partially engaged, fluoroscopy may be used to monitor the advancement of the facet screw to avoid over driving the facet screw into the pedicle. As the distal end 2404 or 2454 of the facet screw 2400 or 2450 approaches the distal end of the pilot hole, partially retract the dilator sheath 2084 so that the head at the proximal end 2408 or 2458 of the facet screw 2400 or 2450 is visible via fluoroscopy. It is recommended that the surgeon use the “two finger tight rule” to hold the driver with only two fingers to avoid over tightening the facet screw.

Step 1108 Remove the instrumentation. Once the facet screw 2400 or 2450 is in place, remove the driver 2130. Next remove the anchored guide wire 2040. Finally, remove the dilator sheath 2084.

Step 1112 Insert second facet screw. Use the midline incision created at step 1004 and repeat subsequent steps to create a tapped pilot hole and deliver the second facet screw.

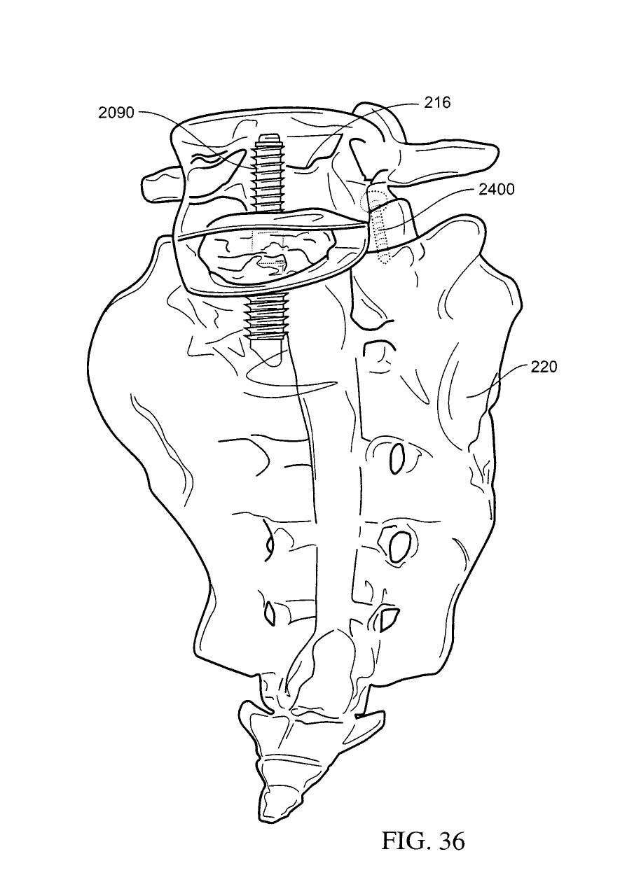

The results of the final process are represented in FIG. 36. FIG. 36 shows a L5/S1 motion segment (216/220) with an implanted fusion rod 2080 and one of the two facet screws 2400 across one of the two facet joints. (Other facet screw across other facet joint not visible in this view).

One of skill in the art will be able to look at the steps set forth in FIG. 5 and make minor modifications within the scope of the teachings of the present disclosure. For example, the act of creating the guide pin assembly in step 1012 could be done before creating the incision in step 1004 but it aids the presentation of the material to teach those of skill in the art to discuss the guide pin assembly creation immediately before the use. The text providing explanation of the various steps and providing detailed information on a way to achieve particular steps should not be misinterpreted as an indication that all of the details provided are necessary in order to practice the method taught by this disclosure. One of skill in the art may make modifications to adjust to personal preferences, patient anatomy, or other factors.

Process Variations and Details.

Some surgeons may opt to not tap the pilot hole so the steps associated with tapping may be considered optional.

In implementations that provide the surgeon a choice of two different facet screw diameters in addition to a set of nominal facet screw lengths, the instrument set may be adapted to have a bore cutter 2200 and facet screw tap 2110 that may be used with both screw diameters by being adequately small to be used with the smaller of the two diameters. This eliminates the risk that the large bore cutter 2200 and facet screw tap 2110 will be used to prepare a pilot hole for a small diameter screw and thus lead to a situation where the facet screw does not achieve a high strength installation.

Details of Components

Facet Screws

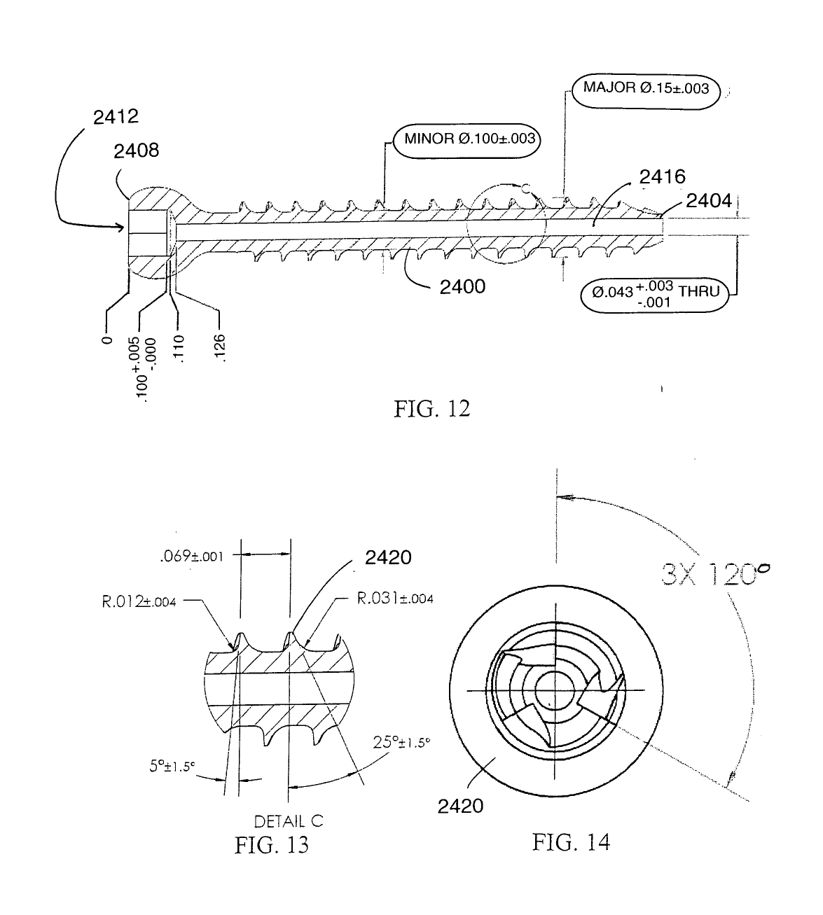

FIG. 12 shows a longitudinal cross section of a fully threaded facet screw 2400. Visible at the proximal end 2408 is the hexagonal shaped opening 2412 to receive a corresponding driver. The fully threaded facet screw 2400 has a cannula 2416 that runs the length of the screw so the screw may be used in connection with a guide wire. FIG. 13 provides an enlarged view of detail C from FIG. 12 to provide details of a thread pattern that may be used for threads 2420 on the fully threaded facet screw 2400. FIG. 14 shows a front view of the distal end 2404 of the fully threaded facet screw.

For both fully threaded facet screws 2400 and lag facet screws 2450, representative dimensions for the example given above are about 25 mm to about 65 mm, often between 25 mm to 40 mm in length, and with major diameters of between about 0.120″ (3 mm) to about 0.196″ (5.0 mm) and often about 4 millimeters to 5 millimeters of major diameter. For example, the surgeons may be provided with a set of pairs of screws in the full range of screw lengths (25, 30, 35, and 40 millimeters) in both 4 millimeter and 5 millimeter major diameters for fully threaded facet screws and another complete set for facet lag screws.

The range of nominal screw lengths may be broader than four nominal sizes and may be longer than 40 millimeters. Some applications may occasionally use screws that are 60 millimeters and even larger. Thus, instrumentation may have more than four distinct landmarks for use with more than four different nominal lengths. While it is likely that an instrument set that is frequently using a screw of a particular nominal length will have visual indicators (landmarks) for use with that nominal length, there may be situations where an unusual screw length may be used that does not have a set of landmarks for that screw length.

Facet screws, as implantable components, may be fabricated from biocompatible orthopedic implant materials that are common medical grade materials with substantial clinical history across a wide variety of orthopedic utilities that present no biocompatibility issues. For example, screws may be formed, machined, preferably from among high strength (high tensile strength, high fatigue strength), wear and abrasion resistant metal alloys (for example, MP35N; Elgiloy,™ a super alloy of cobalt chrome; Co—Cr alloy such as Stellite™; Ti6Al4V alloy, and nitride coated Ti alloys) according to the desired biomechanical properties.

Optionally, the head end of the facet screw may be configured for threaded engagement with a set of external threads from a retention rod included as part of the driver so that the fixation screw can be selectively engaged with the retention rod before insertion into the body and then disengaged from the retention rod after the fixation screw is at least partially inserted in to the bore in the vertebra.

Facet Guide Pin Assembly

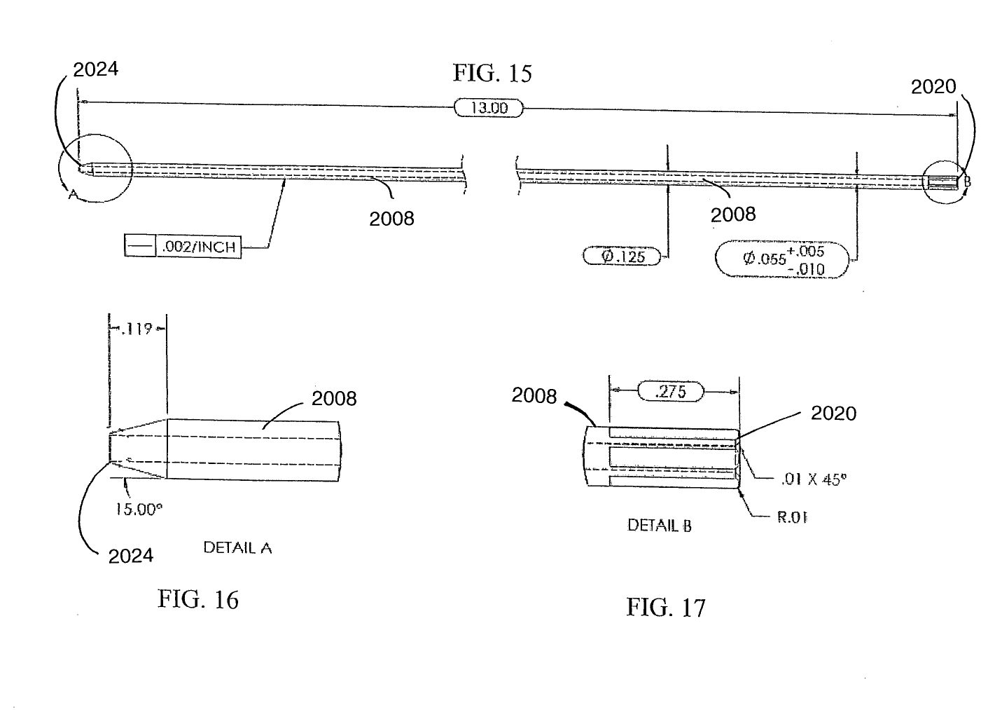

As discussed above, the cannulated guide pin 2008, guide pin with grip 2016, and guide pin handle 2012 are formed into an assembly. Details of these components and subassemblies are provided in FIGS. 15-22. FIG. 15 shows a side view of the cannulated guide pin 2008. FIG. 16 shows the details of the distal end 2024 of the cannulated guide pin 2008. FIG. 17 shows details of the proximal end 2020 of the cannulated guide pin 2008.

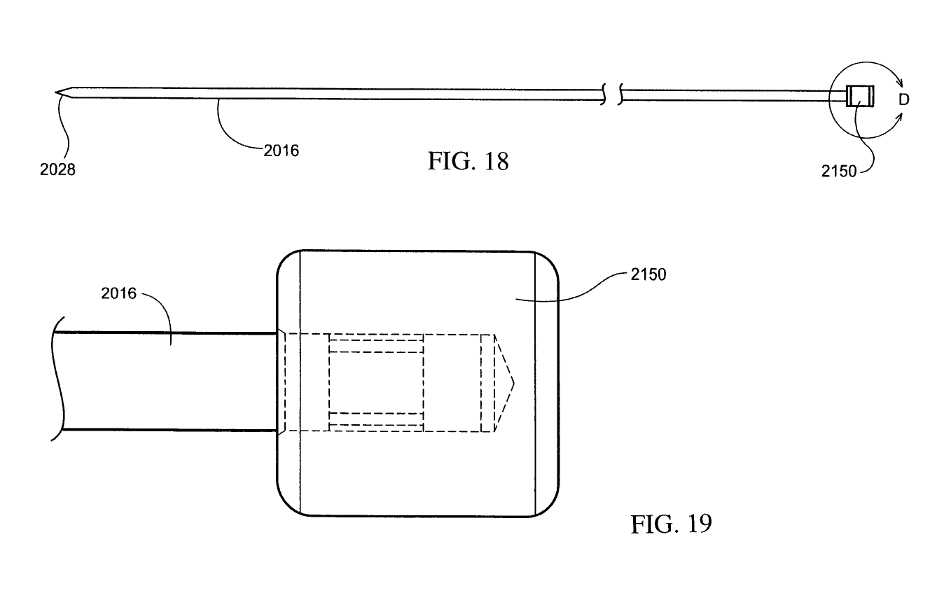

FIG. 18 shows a side view of the guide pin with grip 2016. FIG. 19 provides detail on the grip 2150 at the proximal end of the guide pin with grip 2016. The grip may be affixed to the guide pin with grip 2016 by a press fit and tested by an appropriate pull test. For example the press fit may be tested to ensure that the grip 2150 remains attached while subjected to a five pound pull test.

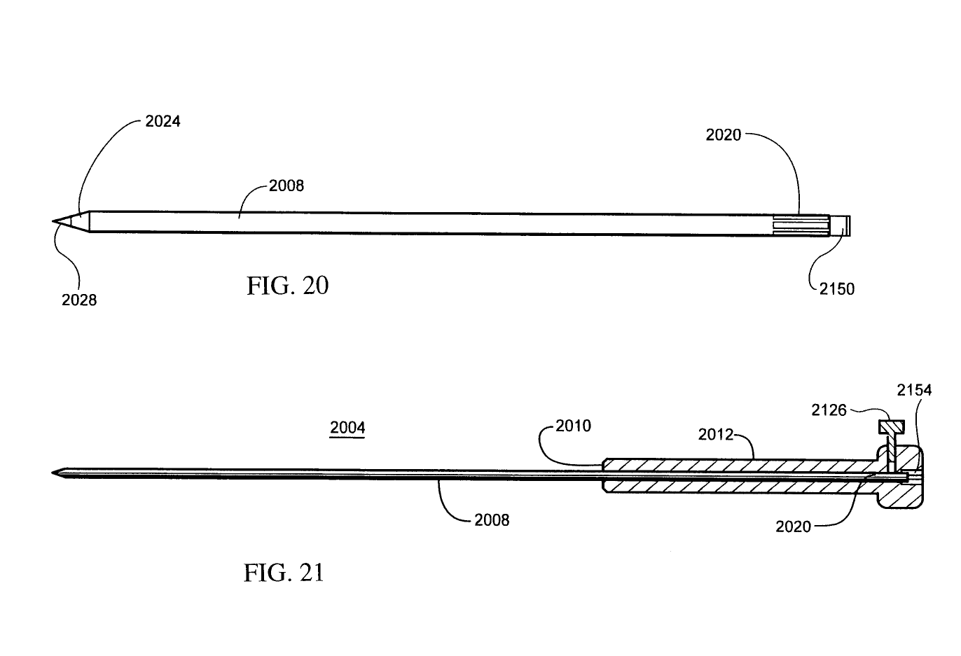

FIG. 20 shows a side view of the subassembly of the cannulated guide pin 2008 with inserted guide pin with grip 2016 visible here only by the distal end 2028 extending beyond the distal end 2024 of the cannulated guide pin 2008 and the grip 2150 extending beyond the proximal end 2020 of the cannulated guide pin 2008.

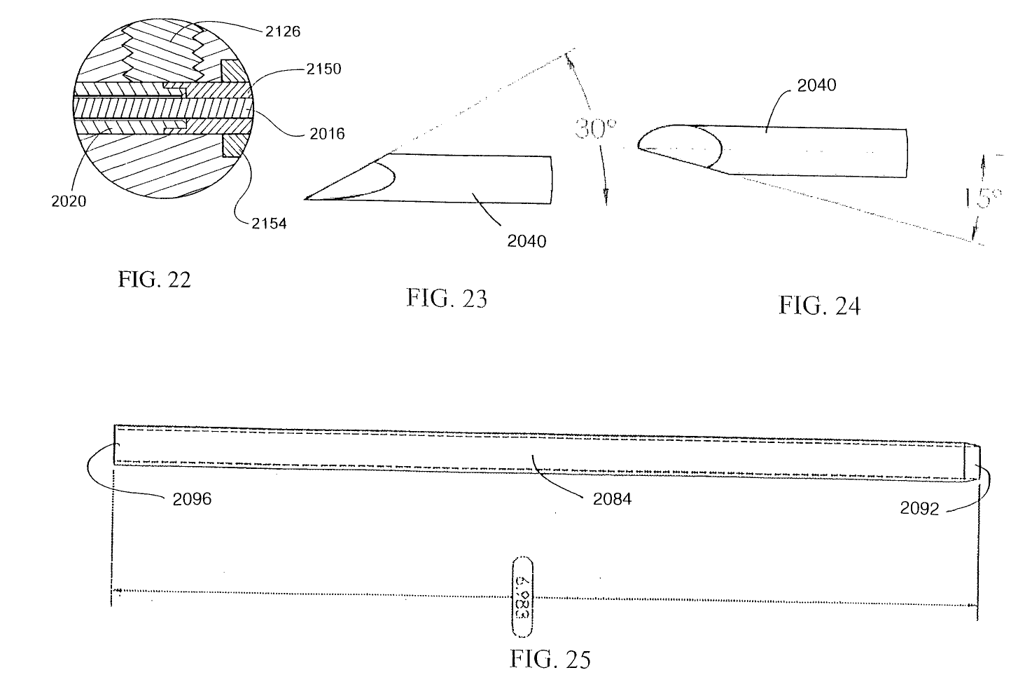

FIG. 21 is a cross section of the complete guide pin assembly 2004 including the guide pin handle 2012 attached via an enlarged set screw 2126. As shown in detail in FIG. 22, the enlarged set screw 2126 engages the proximal end 2020 of the cannulated guide pin 2008 and a portion of the grip 2150 of the guide pin with grip 2016. Also visible in FIGS. 21 and 22 is a female hex fitting 2154 open to the proximal end 2158 of the guide pin handle 2012. The guide pin handle 2012 is used with other instrumentation for other purposes where the female hex fitting 2154 has other uses. The hex fitting is sometimes used to torque the guide pin assembly 2004 to dislodge a cannulated guide pin 2008 from the bone as part of removing the cannulated guide pin.

Guide Wire

FIGS. 23-24 show two views of the distal end 2044 of the guide wire 2040. (See FIG. 9 for an overall view of the guide wire 2040). FIG. 23 shows the first grind which puts a point angle of about 30 degrees. FIG. 24 shows the second grind at about 15 degrees to form a cutting point (as opposed to a cutting edge). The distal end 2044 of the guide wire 2040 must advance through the facet joint and remain anchored in the pedicle during the creation of the pilot hole and delivery of the selected facet screw. Thus, a drilling type end may be used as shown rather than a tip with an apex at the axial centerline of the guide wire (not shown). Note that just as the bore cutter 2200 needs the guide wire 2040 to maintain the trajectory of the bore cutter 2200 and keep if from moving radially away from the intended trajectory, the guide wire 2040 needs the cannulated guide pin 2008 to keep the guide wire 2040 aligned with the intended trajectory and prevent the guide wire 2040 from moving along the surface of the facet joint away from the intended place of entry. After the guide wire 2040 is extended beyond the cannulated guide pin 2008 and into the facet joint, the cannulated guide pin 2008 provides support to the guide wire 2040 to keep the portion not in the facet joint from bending.

The guide wire 2040 may be in the range of 1 millimeter of diameter and 19.75 inches long. While the same guide wire 2040 may be reused to deliver the second facet screw, a kit of single use components for delivery of a pair of facet screws is likely to have a pair of guide wires so that a spare guide wire is available.

Dilator Sheath

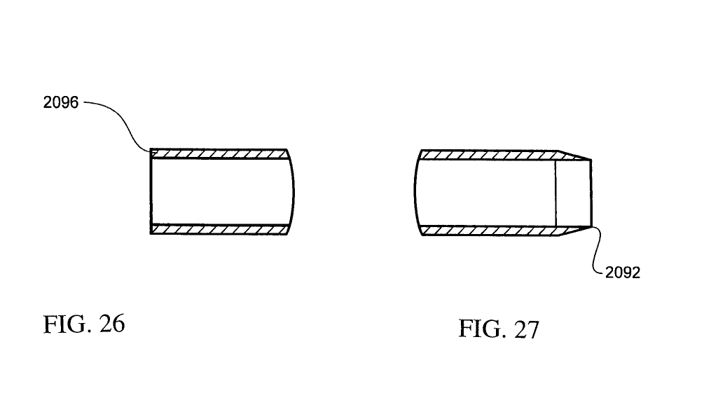

FIG. 25 is a cross section of the dilator sheath 2084. FIG. 26 provides details of the proximal end 2096 of the dilator sheath 2084. FIG. 27 provides details of the distal end 2092 of the dilator sheath 2084.

Bore Cutter

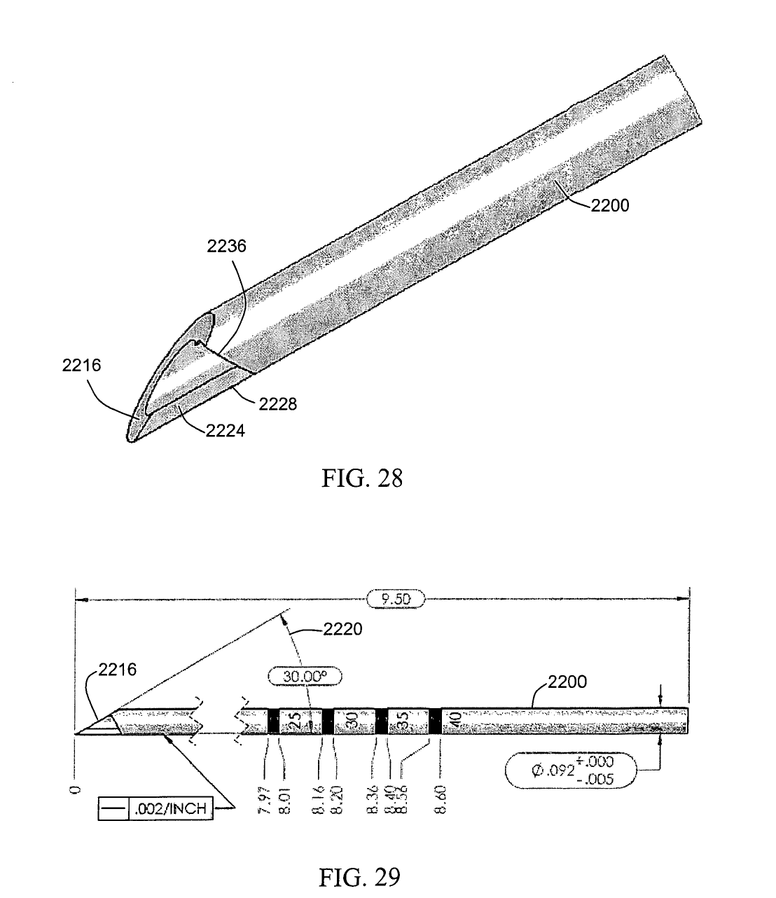

FIG. 28 provides a perspective view of the distal end 2204 of the bore cutter 2200. The bore cutter 2200 is adapted for use with a guide wire 2040 and thus is a tube with outer diameter D and inner diameter d. A first grind called the point grind, creates a first face 2216. The point angle 2220 is show in FIG. 29 as 30 degrees but may be made in the range of about 20 degrees to about 45 degrees. Point angles of less than about 20 degrees would result in a leading “tooth” which was too thin, while a 30 degree bevel results in a sharper tip than a 45 degree bevel.

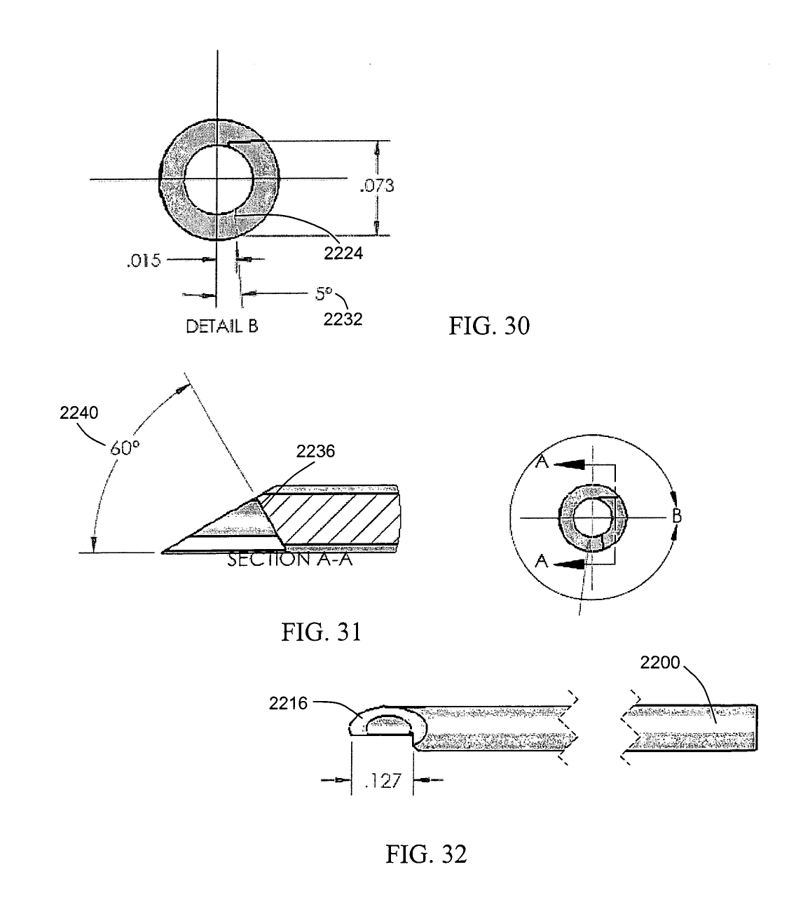

The rake grind creates face 2224 with cutting edge 2228 located on the outer perimeter of the tube (a distance of V2 D from the axial centerline of the bore cutter 2200). While the rake angle could potentially be negative or positive, the range of rake grinds for this bore cutter may range from zero degrees to about twenty-five degrees, in most cases from about 1 degree to 25 degrees. FIG. 30 shows a front view of the bore cutter 2200 from FIG. 28 which has a rake angle 2232 of about 5 degrees. Rake angles less than about 5 degrees yield cutting edges which are less sharp, while angles which are in excess of about 30 degrees approach being blunt on a different plane. The rake grind imparts a leading blade edge on the device.

The clearance grind creates face 2236. FIG. 31 is a cross section taken to show the clearance angle 2240. The clearance angle may range from between about 30 degrees to about 70 degrees, and often is about 60 degrees as is shown in FIG. 31. The clearance angle 2240 helps move cut material towards the outer periphery and away from the cannula of the bore cutter 2200. This reduces backfil. Backfill of bone debris into the cannulated drill is undesirable because “packing” of such debris in the cannula of the bore cutter and against the guide wire 2040 may result in the dislodging or unwanted concomitant removal of the guide wire 2040 (over which the bore cutter 2200 is inserted) during removal of the bore cutter 2200 from the surgical site. The guide wire 2040 needs to remain in place in order to enable the accurate placement of the cannulated facet screws.

While it is thought that a bore cutter 2200 could be used that did not have material removed with a grind for clearance angle, it is currently thought that having this clearance grind is beneficial to the performance of the bore cutter 2200.

The bore cutter described above is unusual in that it may be created from a tube with just two grinds: a first grind to add the point angle to the distal end of the tube followed by a grind to put a cutting edge on the tube by grinding substantially parallel to the axial centerline of the tube to create the cutting edge 2228

FIG. 32 provides a top view of a bore cutter 2200.

The combination of geometry shown in FIGS. 28 to 32 produces a bore cutter that needs an anchored guide wire to restrict the lateral movement of the bore cutter. When restrained by an anchored guide wire having a diameter close to but less than the inner diameter of the bore cutter the combination of angles for the bore cutter minimizes the force required for cortical penetration.

The bore cutter 2200 may be fabricated from a 300 series stainless steel (e.g., medical grade 304 or 316; full hard temper) and is sized to create a pilot hole slightly under the minor diameter and to a depth about equal to the length of the screw (less the head) or device to be implanted.

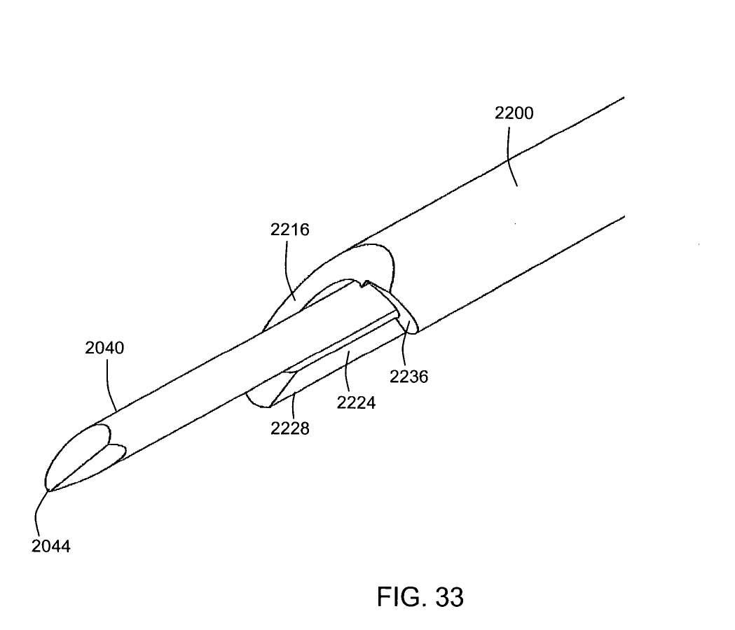

FIG. 33 shows the distal end of bore cutter 2200 with the distal end 2044 of the guide wire 2040. The combination of these two components forms a bore cutter assembly 2250 as the bore cutter 2200 needs the anchored guide wire 2040 for proper operation. The anchored guide wire 2040 serves to limit the radial movement of the bore cutter 2200 and thus center the rotation of the cutting edge (or edges) of the bore cutter 2200 to operation centered in the vicinity of the axial centerline of the guide wire 2040. Note that while the diameter of the guide wire 2040 substantially fills the inner diameter of the bore cutter 2200, there is some clearance. This allows the bore cutter 2200 to rotate relative to an anchored guide wire 2040 without binding against the guide wire 2040. In general, the clearance may be even less than shown here but needs to be positive so that the bore cutter 2200 may move freely relative to the anchored guide wire 2040.

The combination of the guide wire 2040 and the bore cutter 2200 needs to be stiff enough to maintain the intended trajectory of the boring cutter 2200. The teachings of the present disclosure show a way to maintain depth control of the boring cutter 2200 so as to avoid dislodging the anchored guide pin 2040 while creating a pilot hole of an appropriate depth for a screw of an appropriate length.

Typical dimensions for a bore cutter in accordance with the above example are as follows. For example, in drilling a pilot hole about 2 millimeters to 3 millimeters diameter for a screw delivered over a guide wire about 1 millimeter in diameter, a bore cutter is used which (depending on technique used and operative target site) ranges from about 6 inches (152 millimeters) to about 15 inches (380 millimeters) in length, and often about 9.5 inches (240 mm). The outer diameter of the bore cutter may be between about 0.080 inches (2 millimeters) to about 0.180 inches (4.5 millimeters) and often between about 0.087 inches (2.2 millimeters) and about 0.092 inches (2.33 millimeters); and inner diameter of between about 0.040 inches (1 millimeter) and about 0.065 inches (1.5 millimeter) and often between about 0.042 inches (1.1 millimeters) and about 0.058 inches (1.4 millimeters); and with a tube wall thickness of between about 0.014 inches (0.35 millimeters) to about 0.025 inches (0.62 millimeters) and often about 0.020 inches (0.5 millimeters).

One could add another grind to move the cutting edge back from the perimeter of the bore cutter 2200 so that the cutting edge is somewhere between ½ d from the axial centerline to ½ D from the axial centerline of the bore cutter 2200.

While the bore cutter 2200 described within this disclosure has a single cutting edge 2228, nothing in this disclosure should be interpreted as limiting the teachings of the present disclosure to only those bore cutters that have just one cutting edge. One of skill in the art will appreciate that a second or possibly more cutting edges could be cut into the distal end of the bore cutter 2200. It is likely that any additional cutting edge would have an array of characteristics (distance from axial centerline of the bore cutter, point angle, rake angle) that are the same as the first cutting edge, it would be possible to have a first cutting edge as described above and a second cutting edge that has a different array of characteristics so the teachings of the present disclosure do not require that any second cutting edge be the same as the first cutting edge.

Tap

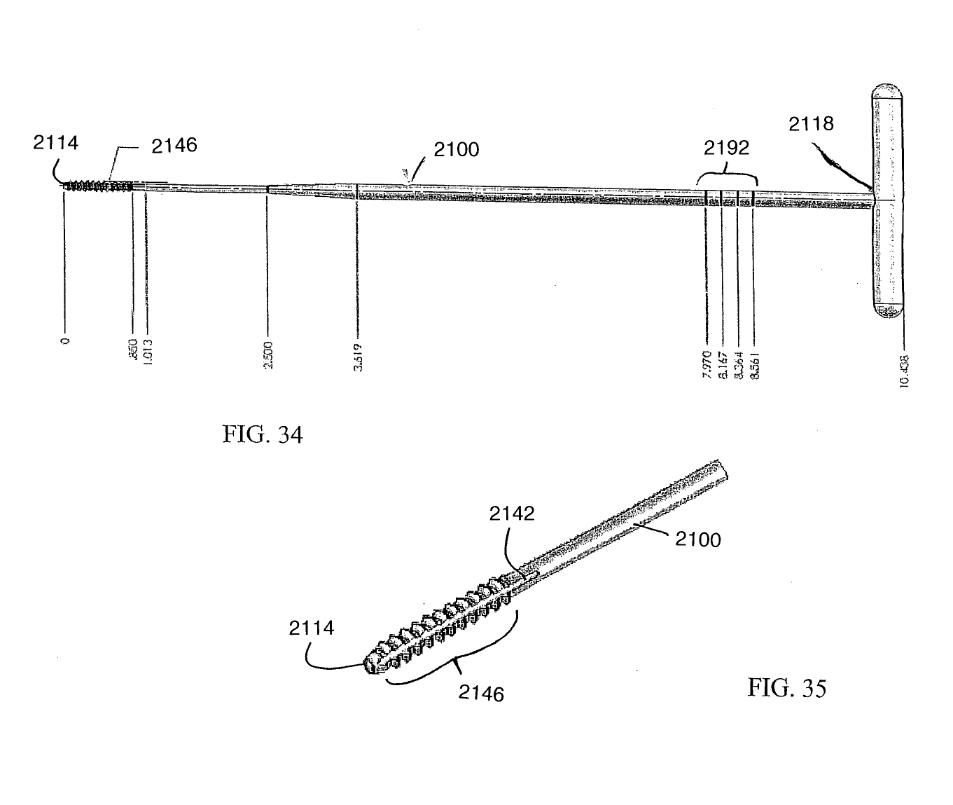

FIG. 34 shows a top view of a facet screw tap 2110 with a set of visual indicators 2122 near the handle 2118 on the facet screw tap 2110. The dimensions in inches are provided as measured from the distal end 2114. As best seen in FIG. 35 showing an enlarged perspective view of the distal end 2114, the tap is cannulated for use over a guide wire. Also visible in FIG. 35 is the cutting section 2146. The tap 2110 is typically fabricated from a hardened stainless steel (such as medical grade 17-4 alloy) with titanium-nitride (Ti—Ni) coated threads and flutes 2142 in the cutting section 2146. The tap 2110 is generally sized to tap the pilot hole to slightly less than the thread (minor) diameter and to a depth about equal to the length of the screw (less the head).

For example, in tapping a pilot hole of about 2 mm-3 mm diameter for a screw delivered over a guide wire about 1 mm in diameter in the lumbrosacral spine, a cannulated tap is used which is between about 7.5 inches (190 millimeters) to about 14 inches (355 millimeters) in length and often about 10.25 inches (260 millimeters) (dependent upon the length of the dilator sheath 2084 (FIG. 6); an outer diameter of the tap between about 0.10 inches (2.5 millimeters) to about 0.375 inches (9.5 millimeters) and often about 0.187 inches (4.75 millimeters); and an inner diameter of between about 0.025 inches (0.63 millimeters) and about 0.135 inches (3.4 millimeters) and often about 0.049 inches (1.2 millimeters). Frequently the tap will be chosen to create a tapped thread major diameter to be smaller than the major diameter of the threads on the facet screw. The major diameter of the thread created by the tap may ranges from between about 0.075 inches (1.9 millimeters) and about 0.30 inches (7.6 millimeters), and is often about 0.130 inches (3.3 millimeters), while the thread minor diameter for the tap is smaller than the drilled pilot hole and ranges from between about 0.050 inches (1.25 millimeters) and about 0.295 inches (7.5 millimeters), and is often about 0.090 inches (2.3 millimeters). The core diameter of the tap is smaller than the thread minor diameter, and ranges from between about 0.045 inches (1.2 millimeters) and about 0.290 inches (7.4 millimeters), and is often about 0.080 inches (2.0 millimeters). The thread length may range from between about 0.20 inches (5 millimeters) and about 2 inches (50 millimeters), and is often about 0.85 inches (22 millimeters). Typically, the tap has between 2 and 4 flutes 2142, often 3, and the flute length extends beyond the cutting section 2146 to facilitate chip removal, ranging from between about 0.25 inches (6 millimeters) to about 2.1 inches (51 millimeters), and is often about 1 inch (25 millimeters).

The tap is advanced by turning the proximal end so that the flutes and threads at the distal end of the tap progressively cut a thread path into the pilot hole bored into the bone to pre-thread a path to facilitate the subsequent insertion of self-tapping (versus self-drilling) facet screws. Note, that since the threads on a partially threaded facet lag screw 2450 must first travel through the proximal end of the pilot hole, the tapping process is the same for both fully threaded facet screws 2400 and lag facet screws 2450. Taps are generally expensive to manufacture given the material that is used to create a tap. Thus, taps are generally not disposable parts and are instead cleaned and sterilized for reuse.

Driver Details

The facet screw driver generally include elongate bodies (or shafts) and handles fabricated from stainless steel alloys, such as those described in ASTM F899-02 Standard Specifications for Stainless Steels for Surgical Instruments or, for example, 17-4 alloy where torque is a consideration, such as when driving components into bone. Similarly, for this reason, as well as to prevent transfer of dissimilar metallic elements to the implant which may contribute to electrochemical corrosion in-situ some driver tips may be fabricated from the same materials as the implantable translaminar facet screws (such as a titanium alloy). As mentioned above, the handle could be a T-handle or a lower profile handle (as shown in FIG. 6) such as normally found on screw drivers.

Scale

The guide wire, boring cutter, tap, and other components are selected based on the range of diameters of facet screw used (in the example above, the facet screw choices were a 4 millimeter major diameter or a 5 millimeter major diameter). If the smallest facet screw to be used was a 5 millimeter screw, then some implementations of the teachings of the present disclosure might be made using a guide wire diameter of more than 1 millimeter. For example if the smallest screw diameter was 5 millimeters rather than 4 millimeters, the system could be set up to use a guide wire with a diameter of 1/16 of an inch. This diameter would be slightly under 1.6 millimeters. As mentioned above, this disclosure does not set an upper limit on the use of guide wire (as opposed to guide pin) at 1.5 millimeters.

Other Motion Segments

The example provided above addressed the placement of facet screws across the L5/S1 facet joints. One of ordinary skill in the art could adjust the example provided above to apply these teachings to deploy a facet screw to another motion segment including other motion segments in the lumbar region of the spine. The adjustments may include changing the scale of the facet screws to match the dimensions of the anatomy and potentially adjusting the dimensions of the various components shown in FIG. 6.

Deployment of Other Fixation Screws

While the example given above was for the deployment of a facet screw across a facet joint, one of ordinary skill in the art could adjust process in accordance with teachings of this disclosure to deliver a bone fixation screw such as a pedicle screw. The dimensions of the screw and of the components in FIG. 6 would be adjusted as appropriate for the specific use.

Use of Specialized Boring Cutters and Taps

While one of skill in the art will appreciate the cost savings in having a series of landmarks on a boring cutter 2200 and on a tap 2110 (if a tap is used in a particular process) so that the boring cutter 2200 and the tap 2110 may be used with a variety of screw lengths, it is not essential that it be done this way. One could enjoy some of the benefits of the present disclosure by having one guide wire 2040 with a series of landmarks 2052 to help identify the appropriate screw length to use so as to leave an adequate amount of the guide wire 2040 anchored in the patient but using a specialized boring cutter (not shown) that has only one landmark and thus is best used with only one particular screw length. Thus, a surgeon would need access to a series of specialized boring cutters (not shown), one for each nominal screw length that is commonly used.

Likewise, instead of having one tap 2110 with a series of landmarks 2122 so that the tap 2110 may be used with confidence for a variety of different pilot hole depths, a surgeon could have access to a series of taps (not shown), with each tap having just one landmark for use with just one screw length.

Finally, since the risk of a tap dislodging the anchored section of the guide wire is less than the risk posed by use of a boring cutter, one could imagine a system where the boring cutter had one or more visual landmarks but the tap did not and the surgeon simply tapped until feeling resistance at the bottom of the pilot hole. The relative position of the distal end of the tap relative to the distal end of the guide wire could also be periodically reviewed via fluoroscopy.

Kits

Various combinations of the tools and devices described above may be provided in the form of kits, so that all of the tools desirable for performing a particular procedure will be available in a single package.

The components described above may be divided into three categories (facet screws, single use components, re-usable components). Kits may be arranged by these three categories. For facet screws, there may be a started kit with a full range of screws (at least two screws of each type) representing all combinations of screw type (full or lag), each of the nominal screw lengths, and repeating the set for each of the available screw diameters. Subsequent replacement sets of screws may be available in sets of two screws.

The reusable components may come in a kit that would include a facet screw tap 2110, a mallet or other impact tool such as a slap hammer 2036, a driver for the facet screws, and a dilator 2100 (FIG. 6) that works with the dilator sheath 2084 provided with the disposable kit. The dilator 2100 may not be provided with a kit if the same dilator 2100 was used in a process to deploy a fusion rod through a trans-sacral access channel in a process that would immediately precede the deployment of the facet screws.

The kit of reusable items might exclude a slap hammer 2036 or mallet as these items may be standard items in the hospital and not need to be in a special kit. The wire driver and power driver used with the bore cutter 2200 are likely to be sold separately from the various instruments shown in FIG. 6. As noted above, the wire driver and the power driver may be the same component and used for both tasks.

The single use components that may be combined together for convenience in a kit. The single use kit may include the cannulated guide pin 2008. The single use kit may include the guide wire 2040 and most likely a second guide wire 2040 so that a second guide wire 2040 is available for deployment of the second facet screw into the second facet joint in the event that the first guide wire 2040 picks up a small bend or other issue from the use deploying the first facet screw. The single use kit may include a dilator sheath 2084 that fits over the dilator 2100 (it is likely that the dilator 2100 which may be a fair amount of metal would be part of the reusable components). The single use kit may include the boring cutter 2200 (some may call this a cannulated bone drill). The single use kit may include the guide pin with grip 2016 and the guide pin handle 2010.

While the initial training of surgeons in this technique may be quite detailed, it is likely that each kit of single use components will include at least some instructions pertaining on the use of the components in the kit in keeping with the intended method of use (such as the method set forth in FIG. 5). The instructions are apt to be written instructions tangible media such as paper but could use or include instructions on other tangible media of any type that allows the instructions to be played or displayed on a device. Examples of other types of tangible media include flash drives and DVD discs but include any tangible media that can be used to convey instructions on a method of use.

One of skill in the art will appreciate that the choice of what parts are single use and what parts are reusable is made partially on the costs to manufacture a single use part versus the cost to manufacture that same part to sustain multiple sterilization cycles and the cost to sterilize the part. Thus, one could decide that a part such as the guide pin handle 2010 which may be made primarily from a polymer and thus be sufficiently inexpensive to be a single use part may be redesigned to be a re-usable part.

Teachings May be Used in Isolation or Combined

One of skill in the art will recognize that some of the alternative implementations set forth above are not universally mutually exclusive and that in some cases additional implementations can be created that employ aspects of two or more of the variations described above. Likewise, the present disclosure is not limited to the specific examples or particular embodiments provided to promote understanding of the various teachings of the present disclosure. Moreover, the scope of the claims which follow covers the range of variations, modifications, and substitutes for the components described herein as would be known to those of skill in the art.

The legal limitations of the scope of the claimed invention are set forth in the claims that follow and extend to cover their legal equivalents. Those unfamiliar with the legal tests for equivalency should consult a person registered to practice before the patent authority which granted this patent such as the United States Patent and Trademark Office or its counterpart.

1. A bore cutter system comprising:
a guide wire;
a tube with an outer diameter of D, the tube having a cavity from a distal end of the tube to a proximal end of the tube, such that the tube may be placed over the guide wire;
the distal end of the tube with at least one cutting edge located a distance of ½ D away from the axial centerline of the tube such that the tube requires an anchored guide wire to restrict the movement of the tube to rotation around an axial centerline of the guide wire within the tube.

2. The bore cutter system of claim 1 wherein the tube has at least one cutting edge that is aligned to be substantially parallel with the axial centerline of the tube.

3. The bore cutter system of claim 1 wherein the guide wire has a diameter of less than 1.6 millimeters.

4. The bore cutter system of claim 1 wherein the guide wire has a diameter sufficiently close to an interior diameter of the tube so that the tube is free to move relative to the guide wire but substantially restricted from radial movement by the guide wire.

5. The bore cutter system of claim 1 wherein the tube:
has a at least one cutting edge including a first cutting edge;
located a distance ½ D away from the axial centerline of the tube;
with a point angle in the range of about 20 degrees to about 45 degrees;
with a rake angle of at least zero degrees.

6. The bore cutter system of claim 1 wherein the first cutting edge has a rake angle in the range of about 1 degree to about 25 degrees.

7. The bore cutter system of claim 6 wherein the first cutting edge has a clearance angle in the range of about 30 degrees to about 70 degrees.

8. The bore cutter system of claim 5 wherein the tube has at least a second cutting edge, the second cutting edge:
located a distance ½ D away from the axial centerline of the tube;
with a point angle in the range of about 20 degrees to about 45 degrees;
with a rake angle of at least zero degrees.

9. The bore cutter system of claim 5 wherein the tube has at least a second cutting edge, the second cutting edge:
located a distance away from the axial centerline of the tube different from the distance of the first cutting edge from the axial centerline of the tube; and
with a point angle different than the point angle for the first cutting edge.

10. The bore cutter system of claim 1 wherein the tube:
has a most distal portion of intact tube for a full 360 degrees around the centerline of the tube and this most distal portion of intact tube has an interior diameter d;
has at least one cutting edge:
located a distance at least ½ d and not more than ½ D away from the axial centerline of the tube;
with a point angle in the range of about 20 degrees to about 45 degrees; and
with a rake angle of at least zero degrees.

11. The bore cutter system of claim 10 wherein the first cutting edge has a rake angle in the range of about 1 degree to about 25 degrees.

12. The bore cutter system of claim 10 wherein the tube has at least a second cutting edge:
located a distance at least ½ d and not more than ½ D away from the axial centerline of the tube;
with a point angle in the range of about 20 degrees to about 45 degrees; and
with a rake angle of at least zero degrees.

13. The bore cutter system of claim 1 wherein the tube has at least one visual indicator to assist an operator of the bore cutter system in creating a pilot hole of an appropriate depth for a screw of a particular length without dislodging the anchored guide wire.

14. The bore cutter system of claim 13 wherein the tube has at least two visual indicators to assist the operator of the bore cutter system in creating:
a pilot hole of a first depth which is adequate for use with a screw of a first length without dislodging the anchored guide wire through monitoring of a first landmark; or
a pilot hole of a second depth which is adequate for use with a screw of a second length without dislodging the anchored guide wire through monitoring of a second landmark.

15. The bore cutter system of claim 1 wherein a distal end of the guide wire has a tip with a cutting edge so the guide wire may be driven with a power driver.

16. A bore cutter system comprising:
a tube, the tube having a cavity from a distal end of the tube to a proximal end of the tube, such that the tube may be placed over an anchored centering means;
a centering means for centering the tube while boring into tissue;
the tube having a cutting means adapted to bore through tissue while rotating relative to the anchored centering means.

17. The bore cutter system of claim 16 wherein the cutting means includes a cutting edge aligned substantially parallel with an axial centerline of the tube.

18. The bore cutter system of claim 16 wherein the tube has at least one visual indicator to assist an operator of the bore cutter system in creating a pilot hole of an appropriate depth for a screw of a particular length without dislodging the anchored centering means.

19. The bore cutter system of claim 18 wherein the tube has at least two visual indicators to assist the operator of the bore cutter system in creating:
a pilot hole of a first depth which is adequate for use with a screw of a first length without dislodging the anchored centering means through monitoring of a first landmark; or
a pilot hole of a second depth which is adequate for use with a screw of a second length without dislodging the anchored centering means through monitoring of a second landmark.

20. The bore cutter system of claim 16 wherein the tube has a tube wall with an inner diameter of d and an outer diameter of D;
the cutting means includes at least one cutting edge located a radial distance of at least ½ d but not more than ½ D from an axial centerline of the tube.

21. The bore cutter system of claim 16 wherein:
the cutting means has at least two different cutting edges, a first cutting edge and a second cutting edge wherein the first cutting edge has a cutting edge parameter array comprising:
mean distance from the axial centerline;
point angle; and
rake angle; and
the second cutting edge has a cutting edge parameter array that is not the same as the cutting edge parameter array for the first cutting edge.

22. A method of delivering a facet screw across a facet joint, the method comprising:
using a cannula for inserting a guide wire across the facet joint to anchor the guide wire and to designate a trajectory and position for the facet screw to be placed, the guide wire inserted an insertion depth;
using visual indicators on the guide wire relative to the cannula, to select a screw length that is shorter than the insertion depth of the anchored guide wire;
placing a cannulated bone drill bit over the guide wire and into a working cannula;
creating a pilot hole of appropriate depth for the selected screw length by monitoring a visual indicator on the bone drill bit as the visual indicator approaches the working cannula;
deploying a cannulated facet screw of the selected screw length over the anchored guide wire through a delivery cannula; and
removing the guide wire and the delivery cannula.

23. The method of delivering a facet screw across a facet joint of claim 22, wherein the cannulated bone drill bit is part of a set of cannulated bone drill bits, with the set including at least one cannulated bone drill bit for each of the nominal screw lengths envisioned for use with this method.

24. The method of delivering a facet screw across a facet joint of claim 22, wherein the cannulated bone drill bit has a set of at least two visual indicators to assist in creating:
a pilot hole of a first depth which is adequate for use with a screw of a first length without dislodging the anchored centering guide wire through monitoring of a first visual indicator; or
a pilot hole of a second depth which is adequate for use with a screw of a second length without dislodging the anchored guide wire through monitoring of a second visual indicator.

25. The method of delivering a facet screw across a facet joint of claim 22, wherein a distal end of the guide wire has a drilling tip so that inserting a guide wire across the facet joint to anchor the guide wire and to designate a trajectory and position for the facet screw to be placed is performed using a power driver to insert the guide wire.

26. A method of delivering a facet screw across a facet joint, the method comprising:
anchoring a guide wire through a first cannula and across the facet joint to designate the position for the facet screw to be placed; the anchored guide wire extending beyond the first cannula by an anchor depth of A+L;
using visual indicators on a proximal end of the guide wire, visible beyond a proximal end of the first cannula to select a screw length of L;
removing the first cannula while leaving the anchored guide wire;
placing a second cannula over the anchored guide wire;
placing a cannulated bone drill bit over the guide wire and into the second cannula with a larger inner diameter than the first cannula to position a distal end of a bone drill bit;
creating a pilot hole of appropriate depth for a screw length of L while not drilling more than L from the distal end of the second cannula by monitoring a one visual indicator on the portion of the bone drill bit exposed beyond the proximal end of the second cannula, the visual indicator corresponding to the desired depth of insertion for a screw of length L in order to leave the anchored guide wire in an anchor depth of at least A;
deploying a cannulated facet screw of the selected length over the guide wire and through a delivery cannula; and
removing the guide wire and the delivery cannula.

27. The method of claim 26 wherein the cannulated bone drill bit has a set of at least two visual indicators so that the cannulated bone drill bit may be used to create pilot holes of appropriate length for a set of at least two different screw lengths while leaving the anchored guide wire in an anchor depth of at least A.

28. The method of claim 26 wherein the delivery cannula is the same cannula as the second cannula.

29. The method of claim 26 wherein the pilot hole is tapped by placing a cannulated tap over the guide wire and through the second cannula and then rotated to tap the pilot hole while not advancing the tap more than L from the distal end of the second cannula by monitoring a visual indicator on a portion of the tap exposed beyond the proximal end of the second cannula, the visual indicator corresponding the desired depth of insertion for a screw length of L in order to leave the anchored guide wire in an anchor depth of at least A.

30. The method of claim 29 wherein the cannulated tap has a set of at least two visual indicators so that the cannulated tap may be used to tap pilot holes of appropriate length for a set of at least two different screw lengths while leaving the anchored guide wire in an anchor depth of at least A.

31. The method of claim 26 wherein first cannula with inserted guide wire are advanced through tissue to select a desired delivery trajectory as a preliminary step in preparing to anchor the guide wire through the first cannula and across the facet joint to designate the position for the facet screw to be placed.

32. The method of claim 26 wherein preliminary steps to prepare to anchor the guide wire through the first cannula and across the facet joint to designate the position for the facet screw to be placed include:
inserting a preliminary guide wire into the first cannula;
advancing the first cannula an inserted preliminary guide wire through tissue to select a desired delivery trajectory; and
removing the preliminary guide wire from the first cannula.

33. A facet screw delivery system kit for a method of delivery of a facet screw via an aligned percutaneous access, the kit comprising:
a cannulated guide pin;
a guide wire with a distal portion to be anchored into bone with visual indicia to indicate appropriate length of facet screw to use so that a pilot hole of appropriate length for a facet screw of a selected length may be prepared while leaving the distal portion of the guide wire anchored into bone;
a dilator sheath that fits over a distal portion of a dilator; the dilator sheath having a distal end to be positioned towards an anchored end of an inserted guide wire and a proximal end; and
a cannulated bone drill for creating a bore, the bone drill having a visual indicia to indicate that the bone drill has been advanced an appropriate amount beyond the distal end of the dilator sheath for a previously determined appropriate length of facet screw to use while leaving an anchored guide wire anchored in bone wherein a visual indicia on the cannulated bone drill corresponding to a visual indicia on the guide wire leads to insertion of a guide wire of a distance of A+L beyond the distal end of the cannulated guide pin and insertion of the cannulated bone drill of a distance L beyond the distal end of the dilator sheath so that the inserted cannulated bone drill is inserted with depth control to maintain an anchored depth of at least A for an anchored guide pin.

34. The facet screw delivery system kit of claim 33 further including a second guide wire that is interchangeable with the guide wire.

35. The facet screw delivery system kit of claim 33 further including:
a guide pin with grip; and
a guide pin handle.

36. The facet screw delivery system of kit claim 33 further including instructions on tangible media for a method of delivering a facet screw across a facet joint, the method comprising:
using a cannula for inserting a guide wire across the facet joint to anchor the guide wire and to designate a trajectory and position for the facet screw to be placed, the guide wire inserted an insertion depth;
using visual indicators on the guide wire relative to the cannula, to select a screw length that is shorter than the insertion depth of the anchored guide wire;
placing a cannulated bone drill bit over the guide wire and into a working cannula;
creating a pilot hole of appropriate depth for the selected screw length by monitoring a visual indicator on the bone drill bit as the visual indicator approaches the working cannula;
deploying a cannulated facet screw of the selected screw length over the anchored guide wire through a delivery cannula; and
removing the guide wire and the delivery cannula.

37. A facet screw delivery system kit for a method of delivery of a facet screw via an aligned percutaneous access, the kit comprising:
a cannulated tap for use over an anchored guide wire for tapping a previously created pilot hole, the tap having a visual indicia to indicate that the tap has been advanced an appropriate amount beyond the distal end of a cannula tap the previously created pilot hole for a screw of a particular length while leaving the anchored guide wire anchored in bone; and
a cannulated driver for use over an anchored guide wire, the driver having a distal end with a driving engagement section for engaging with a corresponding driving engagement section on a facet screw.

38. The facet screw delivery system kit of claim 37 further comprising a cannulated dilator for use over a guide wire, the cannulated dilator adapted for use with a dilator sleeve.
